(19) United States
(12) Patent Application Publication
(10) Pub. No.: US 2007/0203747 A1
(43) Pub. Date: Aug. 30, 2007

(54) E-COMMERCE SYSTEMS AND METHODS

(75) Inventors: Siamak Baharloo, Carlsbad, CA (US); James Gilmore, Del Mar, CA (US); Michael Stapleton, San Diego, CA (US); Anthony Stevens, San Diego, CA (US); Martin Naley, East Amherst, NY (US); Nicole Cheri Brockway, Lewiston, NY (US)

Correspondence Address: INVITROGEN CORPORATION;C/O INTELLEVATE, P.O. BOX 52050, MINNEAPOLIS, MN, 55402, US

(73) Assignee: INVITROGEN CORPORATION

(21) Appl. No.: 11/567,726

(22) Filed: Dec. 6, 2006

Related U.S. Application Data
(60) Provisional application No. 60/742,543, filed on Dec. 6, 2005.

(51) Int. Cl. G06Q 50/00; G06Q 10/00; C12Q 1/00

(52) U.S. Cl. 705/2; 435/4

(57) ABSTRACT

The invention is in general directed to methods and processes for generating revenue via an electronic commerce website. The invention is also directed to methods of processing product orders and selling product related to protocols, workflows, or other applications. The invention also contemplates methods for selling products, which include previously ordered products, related products, substitute products, and equivalent products, including but not limited to equivalent protocol products, matched reagent sets, kits, and biomodules for practicing said protocols and equivalent protocols.

This application claims benefit of priority to U.S. provisional application 60/742,543, filed Dec. 6, 2005, which is incorporated by reference herein in its entirety.

### BACKGROUND OF THE INVENTION

1. Field of the Invention

The invention is in general directed to methods and processes for generating revenue via an electronic commerce website. The invention is also directed to methods of processing product orders and selling product related to protocols, workflows, or other applications. The invention also contemplates methods for selling products, which include previously ordered products, related products, substitute products, and equivalent products, including but not limited to equivalent protocol products, kits, and biomodules for practicing said protocols and equivalent protocols.

2. Background Information

The structure and organization of scientists, collaborators, service providers, and other customers often occur in groups and various subgroups. Administratively, these grouping structures provide problems in making purchases. For example, in a university laboratory setting, a group of scientists may be organized such that all product orders are charged to one particular account. Alternatively, a group of scientists or other customers may be organized according to sub-accounts within the group where purchases may be charged to more than one account, including additional sub-accounts. In some cases, scientists may work under grants funding a particular area of research against which those scientists charge certain purchases. In other cases, one scientist may collaborate with two or more scientists, under more than one research grant. Complex groups can be encountered in a number of work environments, including office, retail, scientific, and service environments. The complexity and various permutations encountered in these collaborative work environments lead to difficult and inefficient purchasing processes. As should be readily understood, managing product purchases, either according to the particular customer within a group or the accounts against which those purchases should be charged, is cumbersome.

Some vendors have been known to provide product onsite in a storage facility or other onsite location so customers may obtain product. Typically the storage facility is stocked with a limited number of products in varying quantities. When needed, customers take product from the storage facility and the transaction is recorded. The individual may charge the product to different accounts. This method is extremely limited and does not address the complexity of group ordering dynamics in that it requires vendors to provide product in advance of purchase, requires a physical location onsite for the vendor's product, requires restocking of individual storage facility at every onsite location, and is generally not capable of providing a full inventory of products.

With the increasing popularity of computers (for example, personal computers including smaller devices with computing ability) and advancements in telecommunication network technology, many industries have used these new innovations to improve many commercial operations. In the retail-merchandising arena, for example, hosts of products such as books, music, electronics, athletic gear, etc. are available for online purchases through the Internet. By effectively utilizing virtual stores, merchants streamline purchasing and delivery process for both the consumer and retailer. In similar fashion, telecommunication networks make it possible for many other industries to conduct business in a more efficient manner. To name just a few examples, industries taking advantage of such innovations are financial institutions, travel agencies, and news/media networks. In short, a wide range of industries benefit from the use of computer technology to improve communications, regulatory compliance, manufacturing schedules, security, marketing, sales, and distribution of products and information.

As such, the World Wide Web (WWW) has become a significant new medium for commerce, which is referred to as electronic commerce or E-commerce. Vendors offer goods and services for sale via various WWW sites. However, many of the initial WWW systems were not interactive, and typically addressed only ongoing relationships previously worked out manually, for which extremely expensive custom systems needed to be developed at buyers' or vendors' sites.

Extranet Web technology has been developed to enable a corporation to “talk to” its suppliers and buyers over the Internet or otherwise secure communication routes as though the other companies were part of the corporation's internal “intranet.” This information exchange is done by using, for example, client/server technology, Web browsers, and hypertext technology used in the Internet, on an internal basis, as the first step towards creating intranets and then, through them and connections to the outside, extranets.

For corporations that sell and distribute at wholesale or retail, one technique for selling goods over the Internet uses the concept of a catalog Website that enables buyers to browse through Web pages and use a “shopping cart” feature for selecting items to purchase. Most of these catalog Websites are significantly limited in the interaction, if any, they allow between buyers and sellers (e.g., U.S. Pat. No. 5,117,354). Many corporations, such as General Electric and General Motors, use electronic communications for soliciting bids and ordering parts, supplies, raw materials, products and services on a wholesale basis. The present system and methods are amenable to any scale and any stage of providing information and ordering products and/or services.

Many vendors of biologically related products have also taken advantage of E-commerce to sell goods and services to buyers. Scientists, as consumers of such products, may be interested in more information about a particular product's characteristics beyond availability and price, to include biological attributes such as sequence similarity, linkage data, metabolic and signal pathway participation, compatibility with other systems or molecules, alternative pathways for substrate or product (and availability or provision thereof), etc. Scientists may also be interested in determining the availability of all of the products that are related to their area of research, for example, all of the products that might be used to determine a gene's expression and function, for example, products that could be used to determine the phenotype of cells in which the gene's expression is inhibited or overexpressed, the effect of particular candidate drug molecules on the gene or protein it encodes, or protein/protein interactions within a biological pathway of which the target protein is a member.

Internet vendors have sometimes linked various related products so that if a customer selects one item, the customer is presented with a list of other items that the vendor sells that are related to that selected item. For example, if a scientist selects a type of electrophoresis gel, the scientist may be offered a list of other products, such as buffers, dyes, or molecular weight standards, that the scientist may be interested in purchasing. Also, the scientist may be offered a list of products all related to the same biological application or category, for example, all of the products that may be used to perform gene expression research. These products may include kits that can be used in certain aspects of some biological applications.

Because electronic commerce allows vendors to reach a virtually unlimited number of customers with a vendor's full product line and is particularly well suited for selling products to groups. Typically, each end user of an E-commerce website is provided log on information unique to that user. The E-commerce system then associates product order transaction data with the user, in many cases by correlating the users' log on information to the product order transaction data. A shopping cart is provided within many E-commerce websites that contains product order transaction data for a particular end user. This model, however, is inefficient and lacks flexibility when used by customers or end users in a group setting.

Accordingly a need exists for E-commerce website systems and methods for processing product orders and purchasing services to accommodate group and subgroup customers and end users.

Furthermore, the variety of products offered for sale by vendors and needed by customers can be large. For example, discoveries of new medical diagnostics for diagnosing and prognosing a medical condition, and new medical treatments for treating these medical conditions, including new pharmaceuticals, requires years of medical, biological, and biochemical research. This research continues to become more powerful and accelerated by the discovery and availability to scientists and physicians, of a huge number of increasingly powerful research tools and huge amounts of biological information that is being obtained using these research tools. The research involves numerous procedures and assays that involve the preparation or purchase of a multitude of biological research products. The various procedures and assays often can be grouped into what can be termed a “workflow” that is a group of biological assays and procedures that all may be performed to achieve a certain biological research goal. The various assays and procedures may be performed, as needed, in series or in parallel. Thus, for one workflow, for example, a workflow designed to conduct gene expression analysis, a multitude of biological research products used for the assays and procedures must be obtained, either by preparing or purchasing the products. The research tools include, for example, biological research products, services, protocols, and instruments, as well as isolated biomolecules. With this availability of a growing number of research tools and huge amounts of biological and medical information, it is more difficult for scientists and physicians to be aware and knowledgeable of all of the research tools and biological and medical information available to them.

Similarly, scientists and researchers use certain protocols that have been tested, used, and reported by the scientific community. These protocols take many forms but in general provide the steps or instructions on accomplishing a goal, such as analyzing a sample, creating a research tool, identifying a biological product, etc. Typically, the protocol lists the required steps to accomplish the intended goal and will often require a number of different products to execute the steps of the protocol. Customers will purchase product necessary to execute the protocol after determining the appropriate protocol for their desired goal. Currently, no known system, method, or device exists that facilitates the purchase of products from a single vendor to execute substantially all steps of a well-established protocol. Additionally, when purchasing products for a protocol from one or more vendors, there is no information concerning whether the products used in the protocol are compatible with each other.

The present invention satisfies these needs and provides additional advantages.

### SUMMARY OF THE INVENTION

Provided herein are methods for generating revenue, comprising providing an electronic representation of a protocol, wherein the electronic representation provides or is juxtaposed with a means for purchasing a product, wherein the product is used in at least one step of a protocol. The method in exemplary embodiments provides an electronic commerce system that provides a protocol on a display, in which the display also provides a direct or indirect electronic means for purchasing a product useful in performing one or more steps of the protocol.

Also provided herein is an electronic commerce system in which more than one end user shares a shopping cart. The electronic commerce system has one or more end user terminals connected to a network and one or more computer systems connected to a network. The computer system has processing units, memory, and/or network interface devices and is configured to transmit and store product data and user data. The computer system is configured to correlate multiple end users to a single shopping cart.

Also provided herein are methods of processing product orders from an electronic commerce website. The method comprises creating a shopping cart with a unique identifier; providing at least one customer with security privileges, associating at least one customer with security privileges to the uniquely identified shopping cart, allowing at least one customer with said security privileges to associate additional customers to the uniquely identified shopping cart and storing product order transaction data from a customer in association with the unique identifier of a shopping cart.

Also provided herein are methods for generating revenue comprising, providing at least two customers access to a data entry function for entering and storing desired product data, wherein the data entry function is associated with a product ordering function, and wherein product data entered by more than one user is transmitted at the same time into the product ordering function.

Also provided herein are methods of increasing sales for products related to a protocol comprising the steps of providing users with a protocol, associating one or more products with one or more steps of the protocol, and providing users with a means to order one or more products associated with one or more steps of the protocol.

### BRIEF DESCRIPTION OF THE DRAWINGS

FIG. 1 is an illustration of a networked computer system.

FIG. 2 is a screenshot of one embodiment of an electronic commerce system.

FIG. 3 is a representation of one embodiment of a shared shopping cart.

FIG. 4 is a representation of another embodiment of a shared shopping cart.

FIG. 5 is a representation of another embodiment of a shared shopping cart.

FIG. 6 is a screenshot of one embodiment of an electronic commerce system.

FIG. 7 is a screenshot of one embodiment of an electronic commerce system.

FIG. 8 is a screenshot of one embodiment of an electronic commerce system.

FIG. 9 is a screenshot of one embodiment of an electronic commerce system.

### DETAILED DESCRIPTION OF THE INVENTION

Before the present invention is described, it is understood that this invention is not limited to the particular methodology, protocols, and systems described as these may vary or be substituted arbitrarily as desired. It is also to be understood that the terminology used herein is for the purpose of describing particular embodiments only, and is not intended to limit the scope of the present invention which will be described by the appended claims. Many embodiments of the present invention are described in relation to biological protocols, workflows, products, and environments. While many aspects of the present invention are well-suited for implementation in such environments, it will be readily apparent to one of skill in the art that other environments, products, workflows, and protocols may similarly be used in the methods and processes described herein.

It must be noted that as used herein and in the appended claims, the singular forms “a”, “an”, and “the” include plural reference unless the context clearly dictates otherwise. Thus, for example, reference to a “a subset” includes a plurality of such subsets, reference to “a nucleic acid” includes one or more nucleic acids and equivalents thereof known to those skilled in the art, and so forth.

Unless defined otherwise, all technical and scientific terms used herein have the same meanings as commonly understood by one of ordinary skill in the art to which this invention belongs. Although any methods and systems similar or equivalent to those described herein can be used in the practice or testing of the present invention, the methods, devices, and materials are now described. All publications mentioned herein are incorporated herein by reference for the purpose of describing and disclosing the processes, systems and methodologies which are reported in the publications which might be used in connection with the invention. Nothing herein is to be construed as an admission that the invention is not entitled to antedate such disclosure by virtue of prior invention.

As used herein, the term “provider” refers to any individual, institution, corporation, privately owned company, university, or organization seeking to provide products and services.

As used herein, the term “customer” refers to any individual, institution, corporation, university, or organization seeking to obtain genomic and proteomic products and services.

As used herein, the term “subscriber” refers to any customer having an agreement with a provider to obtain public and private genomic and proteomic products and services at subscriber rates.

As used herein, the term “non-subscriber” refers to any customer who does not have an agreement with a provider to obtain public and private genomic and proteomic products and services at subscriber rates.

As used herein, the phrase “related biological product or service” refers to a product or service that relates to a region of a biomolecule, or an entire biomolecule, presented to a customer. The related product or service is typically used to study a biomolecule and can be related to the biomolecule based on, for example, a biomolecular class of the biomolecule. Related biological products and services include, for example, services, equipment, apparatuses, devices, materials, reagents, and compositions used to study a biomolecule, including a region of the biomolecule. Such products and services include, without limitation: transgenic animal services, cell line construction services, cloning services, bioinformatics services, protein-protein interaction services, protein expression or production services, labeling services, assay services, drug testing services, detection services, such as but not limited to immunoassay services and nucleic acid amplification services; and further include compositions such as cells, cell growth media, media supplements, antibiotics, transfection agents, vectors (e.g., plasmids, episomes, artificial chromosomes, phage, viruses), nucleic acid molecules (e.g., ORFs, cDNAs, cDNA fragments, primers, linkers, gene regulatory sequences, DNA methylation sequences, RNAs, antisense RNAs, RNAi molecules or constructs for producing antisense RNA molecules or RNAi molecules, genomic DNA, genomic DNA fragments, nucleic acid libraries including genomic libraries, cDNA libraries, expression libraries, phage display libraries, mutation libraries, fusion construct libraries, aptmer libraries, etc), peptides, polypeptides, antibodies, antibody fragments, detectable labels (including but not limited to: fluorophores, chromophores, fluorescent nanocrystals or “Quantum dots”, heavy isotopes, radioactive nucleotides, and enzymes), resins, matrices, gels, beads, membranes, filters, slides, plates, chips, arrays (e.g., nucleic acid or protein arrays), buffers, protein solubilizers, cell lysis reagents, enzymes, reagents, substrates, columns, filtration units, dialysis units or devices, tubes, tips, tube racks, gel electrophoresis apparatuses, gel blotting apparatuses, nucleic acid amplification devices, nucleic acid or protein quantitation devices, plate readers, spectrophotometers, and kits that includes any of the above.

As a non-limiting example, if a target biomolecule is a protein, then a related product or service can be a polyacrylamide gel for studying the protein, or a kinase substrate identification assay for determining whether the target biomolecule is a substrate for a kinase. Furthermore, the related product or service can be identified not only based on a biomolecular class of the biomolecule, but also, based on one or more specific attributes of the target biomolecule. For example, a polyacrylamide gel related to a biomolecule that is a protein, can be a specific formulation of gel depending on the size of the target biomolecule, for example a 10% polyacrylamide bis-tris gel. Furthermore, the related product or service can be related specifically to the identified biomolecule. For example, where the identified biomolecule is P53, a related product or service can include an antibody against p53, one or a set of siRNAs or RNAi constructs against P53, a clone encoding P53, a transgenic animal mutated in the P53 gene, one or more kinases that phosphorylate P53, or one or more proteins that bind P53. A directly related product or service is a product or service that relates to an entire biomolecule presented to a customer. For example, if an in silico vector design experiment is design of a primer, then a link to a service for synthesizing the primer presented to the customer by the in silico primer design function, is a directly related product.

As used herein, the phrase “indirectly related biological product” refers to a product that relates to a region or feature of a biomolecule presented to a customer, or can be used in the study of a biomolecule, but is not an entire biomolecule presented to a customer. In one embodiment of the invention, an indirectly related product refers to a portion or feature of the entire biomolecule, but the product is less then the entire biomolecule. In another embodiment, the indirectly related product may be peripheral to the specifically identified biomolecule, but related to the identified biomolecule in the sense that the product or service is useful and/or necessary in accomplishing the ultimate experimental goals of the researcher. For example, in an in silico vector design experiment, a link to an indirectly related product may be a link to the purchase of an antibiotic that corresponds to an antibiotic resistance gene that is on a vector that is designed by the in silico biotechnology experiment design and simulation function. Table 2 of U.S patent publication 20060100788 (U.S. patent application Ser. No. 11/112,933, herein incorporated by reference), is a listing exemplary features and associated products. From the specific product listing, general classes of products are revealed that can be used with the methods provided herein.

The phrase “indirectly related biological service” refers to a service that relates to a step, biomolecule, portion of a biomolecule, or feature of a biomolecule, provided by an in silico design or simulation experiment, but is not an entire step of the in silico design or simulation experiment that resulted in the presentation of the service to the customer. Furthermore, an indirectly related service can be related to a region of a biomolecule presented to a customer by the in silico design and simulation function, but is not synthesis of the entire biomolecule present to the customer.

As used herein, a “biomodule” is a group of components designed to perform a workflow application or more than one workflow application. A biomodule component may include, for example, any biological research product or reagent, including, for example, a kit, cell culture media, electrophoresis products, antibodies, purification reagents, and the like.

By “biological workflow” is meant a set of biological applications designed to carry out a particular type of biological research goal, such as, for example, a workflow to study gene expression, protein expression, or to carry out cellular analysis.

By “workflow pod” is meant a set of related biological applications in a workflow.

By “biological application” is meant a type of biological experiment or biological preparation that may, for example, be part of a workflow. A biological application often comprises more than one step or process, and may comprise at least one assay. A biological experiment may include, for example, a biological assay. A biological application may include, for example, the isolation of a protein, isolation of a nucleic acid, or cell culture. For example, each of the following may be considered a biological application: detecting the presence of proteins using a Western blot, detecting the presence of proteins directly in a gel, analyzing gene expression using microarrays, isolating proteins, cloning a gene and expressing it in eukaryotic or bacterial cells, analyzing protein-protein interactions by affinity bead isolation, analyzing protein-protein interactions using a protein array, analyzing cell viability, analyzing apoptosis, performing immuno-cytochemistry, performing reverse transcription-polymerase chain reaction (RT-PCR), microarray analysis, using RNAi to knock-out or knock-down gene expression.

By “validate,” in the context of a biomodule, protocol, or product that has been validated, is meant that the set of components have been tested in combination and shown to cooperatively perform all of the steps of the application using an internal control, or its derivative, wherein the derivative is derived from the steps of the application itself.

As used herein, “appropriate,” including grammatical variations thereof, means capable of being acted on or carrying out an act. For example, an appropriate request or command when inputted into a dialog box would trigger a search of a database to find or identify an object conforming to the request or command (e.g., keyword search to retrieve objects containing the inputted keyword).

As used herein, “electronic storage medium,” including grammatical variations thereof, means space in electronic memory where information is held for later use. For example, this may include, but is not limited to, magnetic tape, CD-ROMS, DVD, optical disks, flash drives, RAM or floppy disk.

As used herein, “electronic inventory,” including grammatical variations thereof, means a digital catalog which corresponds to some or all of the products and or services offered by the vendor.

As used herein, “target item,” including grammatical variations thereof, means data or files to be affected by an action. For example, a target item can be a file name, a word, an image, a text string, a number or a value stored on electronic media that is retrievable upon request by a user.

As used herein, “interfacing,” including grammatical variations thereof, means the method of interaction between a person and a computer, or between a computer and a peripheral device, or between two computers. In a related aspect, user interface would include the environment that permits one to interact with a computer (e.g., World Wide Web, WiFi, browsers, web pages).

As used herein, “user,” including grammatical variations thereof, means an entity that requests services from a server. The entity can be a human or a device (e.g., see input devices, above). A user may also be referred to as a customer or end user.

As used herein, “user terminals,” including grammatical variations thereof, means a node or hardware that accesses a server. User terminals may also be referred to as end user terminals or customer terminals.

As used herein, “bi-directional communication,” including grammatical variations thereof, means a process by which information is exchanged between two systems in both directions, where each system receives and sends information.

As used herein, “searchable,” including grammatical variations thereof, means the ability of data or files to be looked into in an effort to mark, find or discover such data or files.

As used herein, “request,” including grammatical variations thereof, means one or a series of user inputs or commands for retrieving information from a server or database. Request is also used to refer to input by a user of an electronic commerce system that is entered into a data entry function.

As used herein, “inputting,” including grammatical variations thereof, means the act of entering a request or data. For example, typing at a keyboard pointing, speaking to, etc.

As used herein, “hierarchal menu output,” including grammatical variations thereof, means a list transmitted to the user (e.g., but not limited to, a display on a computer screen) of available alternatives for selection by the operator or user organized into orders or ranks each subordinate to the one above it.

As used herein, “display,” including grammatical variations thereof, means what a user sees on a cathode ray tube (CRT) unit or monitor. More broadly, substitutes may be used as displays, such as auditory signals for the visually impaired or any other means of information communication.

As used herein, “subset,” including grammatical variations thereof, means a set each of whose elements is an element of an inclusive set.

As used herein, “graphic user interface (GUI),” including grammatical variations thereof, means a user interface to a computer that uses icons to represent items, such as documents and programs, that the user can access and manipulate with a pointing device or other signal transducer.

A “display” as used herein, refers to the image presented to a user on a display device, for example, a computer monitor or screen. The display can be, for example, a web page. The display can include text, symbols, pictoral images, etc., and can have one or more sections that can be independently opened, closed, expanded, or reduced on the display.

As used herein, “links,” means a point within an electronic representation that directs a user to either another document or other information that may be used by a user.

As used herein, “hyperlinks,” including grammatical variations thereof, means a pointer within a hypertext document that points (links) to another document, which may or may not be a hypertext document.

As used herein, “server,” including grammatical variations thereof, means a functional unit that provides shared services to workstations/clients/users over a network; for example, a file server, a print server, a mail server. The server may be internal or external, single or multitask.

As used herein, “Web page browser,” including grammatical variations thereof, means a program used to read a file or to navigate through a hypermedia document.

As used herein, “module,” including grammatical variations thereof, means, a self-contained functional unit which is used with a larger system. For example, a software module is a part of a program that performs a particular task.

As used herein, “protocol” refers to a plan for the study of a goal or the execution of steps required to conduct an application. A protocol may be made up of a number of discreet steps, and/or subprotocols, and/or applications.

As used herein, “electronic representation” refers to the generation of a visual image in text, symbol, picture, or other form that is associated with data.

As used herein, “research protocol” refers to a plan for the study, execution, or steps required to conduct research.

As used herein, “biological research protocol” refers to a plan for the study, execution, or steps required to conduct a biological application. A biological application may be designed to conduct a particular type of biological research goal, such as, for example, a workflow to study gene expression, protein expression, or to carry out cellular analysis. In exemplary embodiments, a biological research protocol includes sequential steps for conducting a biological application or workflow. In exemplary embodiments, the protocol is directed to achieving an outcome or result, such as nucleic acid transfer into cells or organisms, amplifying a cell or type of cell (i.e., through cell culture methods) or biomolecule (e.g., through synthesis in cells, cell extracts, or by in vitro amplification of nucleic acids); production of a biomolecule or biomolecular complex (for example, through synthesizing proteins, carbohydrates, lipids, or nucleic acids in cell culture system or in vitro by chemical synthesis, in vitro transcription, in vitro nucleic acid amplification, in vitro translation, etc.) separating or substantially separating a cell or type of cell, organelle, cell fraction, molecular complex, or biomolecule; purifying or substantially purifying a cell or type of cell, organelle, cell fraction, molecular complex, or biomolecule; chemically or biochemically modifying a biomolecule; labeling a a cell or type of cell, organelle, cell fraction, molecular complex, or biomolecule; assaying the status or activity of a cell, cell fraction, molecular complex, or biomolecule or the concentration of a chemical moiety or pH of a cell or cell fraction (e.g., by determining pH, calcium concentration, enzyme activity, morphology, or cellular activities such as motility, cytoskeletal rearrangement, axon or dentrite growth, secretion, release of neurotransmitters, etc.), or detecting a cell, cell fraction, molecular complex, or biomolecule. These examples are illustrative and not limiting to the objectives of the various types of protocols that are included in the invention.

As used herein, “well-established biological research protocol” means a scientific procedure used in the biological sciences that has been used in more than 10 scientific publications or more than 10 peer reviewed journals or more than 10 peer reviewed articles.

The term “biological reagents” as used herein generally refers to isolated biomolecules and biological research products utilized in biological research procedures. Biomolecules include but are not limited to various classes of biomolecules, including, but not limited to, proteins, peptides, antibodies, nucleic acids, nucleotides, carbohydrates, and variants of the foregoing, for example. For example, nucleic acids can be RNA, DNA, peptide nucleic acids, “locked nucleic acids”, or other nucleic acid analogues, and can include, but are not limited to, open reading frames, structural genes, transcription units, antisense molecules, RNAi molecules, constructs for expressing antisense or RNAi molecules, primers, linkers, and vectors.

Two target biomolecules are “different” when they are structurally different. For example, two different nucleic acids have different nucleotide sequences. Two different proteins have different amino acid sequences. Biomolecules may be categorized into families or subclasses based on, for example, a function of the related protein or nucleic acid, such as the functions of the proteins presented in, for example, Table 10 of U.S. Patent Application Publication 20060100788 (U.S. patent application Ser. No. 11/112,933), herein incorporated by reference, or, for example, based on the activity of the related protein or nucleic acid, such as those having enzyme classifications (for illustrative purposes only, a protein kinase family may have various subclasses of protein kinases, such as, for example, tyrosine kinases and serine/threonine kinases, each subclass can itself be further subdivided into narrower subclasses). In certain embodiments, the target biomolecule or a protein encoded by the target biomolecule (for example, when the target biomolecule is a nucleic acid encoding a protein) is a signal transduction factor, cell proliferation factor, apoptosis factor, angiogenesis factor, or cell interaction factor. Examples of cell interaction factors include but are not limited to cadherins (e.g., cadherins E, N, BR, P, R, and M; desmocollins; desmogleins; and protocadherins); connexins; integrins; proteoglycans; immunoglobulins (e.g., ALCAM, NCAM-1 (CD56), CD44, intercellular adhesion molecules (e.g., ICAM-1 and ICAM-2), LFA-1, LFA-2, LFA-3, LECAM-1, VLA-4, ELAM and N-CAM); selectins (e.g., L-selectin (CD62L), E-selectin (CD62e), and P-selectin (CD62P)); agrin; CD34; and a cell surface protein that is cyclically internalized or internalized in response to ligand binding. Examples of signal transduction factors include but are not limited to protein kinases (e.g., mitogen activated protein (MAP) kinase and protein kinases that directly or indirectly phosphorylate it, Janus kinase (JAK1), cyclin dependent kinases, epidermal growth factor (EGF) receptor, platelet-derived growth factor (PDGF) receptor, fibroblast-derived growth factor receptor (FGF), insulin receptor and insulin-like growth factor (IGF) receptor); protein phosphatases (e.g., PTP1B, PP2A and PP2C); GDP/GTP binding proteins (e.g., Ras, Raf, ARF, Ran and Rho); GTPase activating proteins (GAFs); guanine nucleotide exchange factors (GEFs); proteases (e.g., caspase 3, 8 and 9), ubiquitin ligases (e.g., MDM2, an E3 ubiquitin ligase), acetylation and methylation proteins (e.g., p300/CBP, a histone acetyl transferase) and tumor suppressors (e.g., p53, which is activated by factors such as oxygen tension, oncogene signaling, DNA damage and metabolite depletion). The protein sometimes is a nucleic acid-associated protein (e.g., histone, transcription factor, activator, repressor, co-regulator, polymerase or origin recognition (ORC) protein), which directly binds to a nucleic acid or binds to another protein bound to a nucleic acid. In certain embodiments, the target biomolecule or the protein related to the target biomolecule is a growth factor receptor, hormone receptor, neurotransmitter receptor, catecholamine receptor, amino acid derivative receptor, cytokine receptor, extracellular matrix receptor, antibody, lectin, cytokine, serpin, protease, kinase, phosphatase, ras-like GTPase, hydrolase, steroid hormone receptor, transcription factor, heat-shock transcription factor, DNA-binding protein, zinc-finger protein, leucine-zipper protein, homeodomain protein, intracellular signal transduction modulator, intracellular signal transduction effector, apoptosis-related factor, DNA synthesis factor, DNA repair factor, DNA recombination factor, cell-surface antigen, hepatitis C virus (HCV) protease or HIV protease.

Not all genes or transcripts encode protein sequences, thus the term “biomolecule” also comprises non-proteins (e.g., lipids, steroids, carbohydrates), and non-protein coding biomolecules such as, for example, various DNA motifs, cis/trans elements, enhancers, DNA methylation sites, non-coding RNA and miRNA.

Biological research products include various types of biological research products, protocols, instruments, and services, including, but not limited to, products such as, for example, cell culture products, detection products, separation media and systems, and microarrays, for example; services, such as, for example, nucleic acid synthesis, vector construction, and performance of one or more assays; protocols such as a protocol for constructing a vector, performing an assay, or making a monoclonal antibody; or instruments such as mass spectrometers, microscopes, or microfluidic devices. Further examples of biological research products include but are not limited to gels, enzymes, buffers, substrates, cofactors, indicator molecules, bioassays, vectors, synthetic nucleic acids (e.g., DNA and RNA primers and pairs of primers), cloning reagents, PCR reagents, cell culture products, and reagents needed for bioassays. Biological reagents are also described in copending U.S. Patent Application Publication 20060100788 (U.S. patent application Ser. No. 11/112,933), incorporated herein by reference in its entirety.

A biological research product or isolated biomolecule, can include, for example, any of the biological research products, services, instruments, protocols, or isolated biomolecules in the collection of biological research products, services, protocols, instruments, and isolated biomolecules available from a commercial biological research reagent, service, and/or instrument provider. A biological research product or isolated biomolecule, can include, for example, any of the biological research products, services, protocols, or isolated biomolecules in the collection of biological research products, services, protocols, and isolated biomolecules disclosed at and linked to the Internet site available on the worldwide web at the URL invitrogen.com, which Internet site is incorporated by reference in its entirety on the date this application is filed, and available in the 2005 catalog of Invitrogen Corporation (Carlsbad, Calif.), which is incorporated by reference in its entirety on the date the priority application to this application was (U.S. provisional application Ser. No. 60/742,543) filed (Dec. 6, 2005), and also the 2006 Invitrogen New Products Catalog available from Invitrogen Corporation (Carlsbad, Calif.; Invitrogen.com), which is incorporated by reference on the date this application is filed (Dec. 6, 2006), as well as the 2005 catalog of Dynal Biotech (Oslo, Norway), which is incorporated by reference in its entirety on the date the priority application to this application was filed, the 2006 Dynal® Product Catalog, and the 2006 Dynal® Microbiology Product Catalog (CD format), both available from Invitrogen Corporation (Carlsbad, Calif.; Invitrogen.com), incorporated by reference in their entireties on the date this application is filed (Dec. 6, 2006). Also incorporated herein is the 2005 catalog of Zymed, Inc. (South San Francisco, Calif., USA) which is incorporated by reference in its entirety on the date the priority application to this application was filed and also the 2006 Zymed® Research Products and Services catalog, the 2006 Gibco® Brand Products Catalog, and the 2006 BioSource™ catalog, all available through Invitrogen Corp. (Carlsbad, Calif.; Invitrogen.com). Also incorporated herein are the Handbook of Fluorescent Probes and Research Products, Ninth edition, by Richard Haugland and edited by Jay Gregory, published by Molecular Probes, Inc. (2002).

“Matched biological reagents” include the following: (i) two or more isolated biomolecules that relate to the same gene; (ii) a combination of one or more isolated biomolecules that relate to the same gene and one or more biological research products that are used to study the gene, (iii) biological research products that are used to study a class of biomolecules and/or a sub-class of biomolecules and optionally one or more isolated biomolecules of the class of biomolecules and/or sub-class of biomolecules and that relate to the same gene, (iv) biological research products that are used in the same or subsequent steps of a workflow and optionally one or more isolated biomolecules studied using the workflow and that relate to the same gene, and (v) biological research products that are used to study a disease and optionally isolated biomolecules that are involved in the disease, such as isolated biomolecules involved in a pathway of the disease. A set of matched biological reagents includes more than one type of matched biological reagent. Fifty sets of matched biological reagents, for example, can include 50 isolated proteins, 50 nucleic acids each encoding a different one of the 50 isolated proteins, and 50 antibodies each recognizing a different one of the isolated proteins. In this example, 3 classes of biomolecules make up one set of matched reagents. The sets, in this example, can be further expanded to include, for example, biological research products, such as 2 types of biological research products. The biological research products can be, for example, research products that are used to analyze proteins (e.g., protein gels) and/or research products that are used to analyze nucleic acids (nucleic acid gels) and/or research products that include antibodies (enzyme-linked immunoassay kits). Accordingly, different matched reagent sets can include the same research products. A collection of matched biological reagents includes one or more sets of matched biological reagents. Matched biological reagents also include, for example, phylogenetically related biomolecules. U.S. Patent Application Publication 20060100788 (U.S. patent application Ser. No. 11/112,933), incorporated herein by reference in its entirety, includes further description and examples of matched reagents and matched reagent sets.

Sets of biological reagents can be bundled that relate to the same biological pathway or condition. Thus, for example, where two different biomolecules, for example, kinase A and kinase B, have been implicated as being members of a particular biological pathway, sets of matched biological reagents for each of kinase A and kinase B may be bundled in a collection of matched biological reagents. A suite of matched biological reagents thus includes a collection of two or more sets of matched biological reagents where the sets of matched biological reagents include biomolecules that are members of the same biological pathway, are implicated in the same disease, or are members of the same disease pathway. For example, such a suite may include, set 1 and set 2. Set 1 may comprise, for example, protein kinase A, a nucleic acid encoding protein kinase A, an antibody that recognizes protein kinase A, a protein gel, labeled secondary antibodies, and a bioassay kit that measures protein kinase A activity. Set 2 may comprise, for example, protein kinase B, a nucleic acid encoding protein kinase B, an antibody that recognizes protein kinase B, a protein gel, labeled secondary antibodies, and a bioassay kit that measures protein kinase B activity. It is understood that the components of set 1 and set 2 need not be in parallel. For example, set 2 may comprise different biological reagents matched to protein kinase B, for example, a cell line that expresses protein kinase B, cell culture media, an antibody that recognizes protein kinase B, and an siRNA directed against protein kinase B expression.

As used herein, “product group” refers to a collection or assembly of more than one product. A product group may include a biomodule or a kit. A product group may include biological reagents and sets of biological reagents.

As used herein, “clone collection” refers to two or more nucleic acid molecules, each of which comprises one or more nucleic acid sequences of interest.

As used herein, the term “host” refers to any prokaryotic or eukaryotic (e.g., mammalian, insect, yeast, plant, avian, animal, etc.) cell and/or organism that is a recipient of a replicable expression vector, cloning vector or any nucleic acid molecule. The nucleic acid molecule may contain, but is not limited to, a sequence of interest, a transcriptional regulatory sequence (such as a promoter, enhancer, repressor, and the like) and/or an origin of replication. As used herein, the terms “host,” “host cell,” “recombinant host” and “recombinant host cell” may be used interchangeably. For examples of such hosts, see Sambrook, et al., Molecular Cloning: A Laboratory Manual, Cold Spring Harbor Laboratory, Cold Spring Harbor, N.Y.

As used herein, “equivalent protocol” means a substituted protocol, different than the reference protocol, but by which equivalent results are obtained. Accordingly, an equivalent protocol may differ from the reference protocol but yields substantially the same result.

As used herein, “shopping cart” is an electronic representation that may include product information, user information, product order transaction data, scratch pad information, management information, or other data relevant to commercial transactions. Typically a shopping cart has an information storage function that is accessed by a customer, in which a customer can direct designations of and/or information on products being considered for purchase by the customer or by another user that can access the shopping cart. The shopping cart, in addition to listing or representing products being considered for purchase on a display, can also store and display information about the products, such as but not limited to, the quantity of a product considered for purchase, the weight, size, or volume of the product considered for purchase, and/or other product attributes, such as color, power requirements, compatibility with other product(s), etc. A shopping cart preferably has editing functions such that a user can remove, add, or change the quantity or attributes of a product designated in the shopping cart. Preferably editing is performed through a GUI system that allows the user to modify the contents of the shopping cart by clicking on options and, optionally, entering numbers or text. A shopping cart in preferred embodiments is configured such that a customer can link to a purchasing function, preferably through a GUI.

A “purchasing function” is a function of an electronic commerce system that, when activated by a customer, allows a customer to purchase items (products or services) selected electronically. When a user activates the purchasing system to purchase a product, the purchasing system typically generates an order for the selected items that is routed to the product supplier, and generates a bill for payment for the selected items by the customer or the customer's institution or sponsor. The purchasing function can request payment through electronic fund transfer, which may be mediated by a financial institution, such as a credit card company or bank, based on account information supplied by the user. The purchase function may, based on identifying information provided by the user, directly link to a customer account that is billed by the provider of the electronic commerce system.

As used herein, “network,” refers to a configuration of data processing devices and software connected for information interchange, typically allowing bi-directional communication. As used herein a network may refer to a wired or wireless network, internet, intranet, or other configuration of data processing devices connected for information interchange.

As used herein, “product data” refers to data that identifies a commercial product that is offered for sale.

As used herein, “user data” refers to data that identifies or correlates to a particular user.

As used herein, “correlate” means the reciprocal or mutual relation between two objects, and in some uses herein, includes a one-to-one mapping of two objects.

As used herein, “textual product information data” refers to product data displayed in text form.

As used herein, “product pricing data” refers to data that correlates a product with pricing information.

As used herein, “product availability data” refers to data that correlates a product with availability information.

As used herein, “associating” means the correlation of one object to another. Association may be inherently determined by the properties of an object or may be determined for purposes of executing a particular application or function.

As used herein, “privileges”, “user privileges”, and “security privileges” refers to permissions. Security permissions or privileges specify the type, scope, frequency, or degree of action a user may take. Various types of privileges can be associated with a user and nothing contained herein is intended to limit the different types of privileges a system may provide or require. Nonetheless, commonly used privileges including display privileges (whether a user will be entitled to see a display), product placing privileges (whether a user will be entitled to place product orders), product order privileges (whether a user will be entitled to submit product orders), product order deletion privileges (whether a user will be entitled to delete placed orders), product order quantity modification privileges (whether a user will be able to modify placed orders), end user correlation privileges (whether a user will be able to correlate a second user to a shopping cart), and account management privileges (whether a user will be able to modify account settings including shipping and billing settings) are used herein.

One such product particularly suited to use in the present invention includes biomodules. Biomodules comprise components for performing at least one biological application, wherein the components of the biomodule have been validated for the cooperative performance of the biological application. The biological application may, for example, be a workflow application. The biomodules may also, for example, comprise components for performing more than one biological application, for example, more than one biological application that is part of the same workflow pod.

As used herein, “research institutions” refers to commercial and non-commercial entities that conduct research, including scientific research.

As used herein, “biological research institutions” refers to commercial and non-commercial entities that conduct research in the biological sciences.

As used herein, “unique identifier” refers to data that is correlated to one object uniquely. Thus a uniquely identified object is identified by data that is unique to that object. Said data may take various forms including text, numbers, algorithms, key numbers, object representation, or symbols.

As used herein, “uniquely identified shopping cart” refers to a shopping cart (i.e. a stored file or files related to product order information or other commercial information) that is uniquely identified by data. Said data may take various forms including text, numbers, algorithms, key numbers, object representation, or symbols.

As used herein, “data entry function” refers to computer processes that may take various forms, including a window, input area, or other form by which a user may input data to transmit over a network. The data entry function is capable of transmitting data over a network and receiving data from a network. The data entry function may similarly include additional functions or modules, including but not limited to a search function or display function. Data entry function may refer to one data entry function or multiple data entry functions. Hence, a system employing two data entry functions for a single page in an electronic commerce website may be described as an electronic website with a data entry function.

As used herein, “product ordering function” refers to a computer process that may take various forms, including a window, a file, an object map, a database, an algorithm, or other process that receives input data from the data entry function. The term product ordering function may refer to one product ordering function or multiple product ordering functions. Hence, a system employing two product ordering functions for a single page in an electronic commerce website may be described as an electronic website with a product ordering function.

As used herein, “search function” refers to a computer process that provides the user with the ability to enter text, data, or other input to search a database, file, or other system. The search function, as used herein, refers to any process by which user input is transmitted over a network to conduct a search, including text searches, object searching, or other searching. The term search function may refer to one process or multiple processes. Search functions are also disclosed in U.S. Patent Application Publication No. 2005/0240352 (application Ser. No. 10/830,074) and U.S. Patent Application Publication No. 2006/0100788 (application Ser. No. 11/112,933), both of which are incorporated by reference herein in their entireties.

In exemplary embodiments the present invention is incorporated into an electronic commerce website.

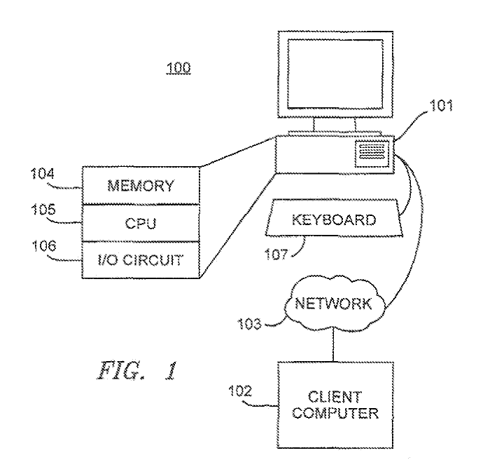

It will be appreciated by one of ordinary skill in the art that computer 101 can be part of a larger system (FIG. 1). For example, computer 101 can be a server computer that is in data communication with other computers. As illustrated in FIG. 1, computer 101 is in data communication with a client computer 102 via a network 103, such as a local area network (LAN) or the Internet.

In particular, computer 101 can include session tracking circuitry for performing session tracking from inbound source to net sale. In one embodiment, as will be appreciated by one of ordinary skill in the art, the present invention can be implemented in software executed by computer 101, which is a server computer in data communication with client computer 102 via network 103 (e.g., the software can be stored in memory 104 and executed on CPU 105), as further discussed below.

The present invention may be implemented using hardware, software or a combination thereof and may be implemented in a computer system or other processing system. In fact, in one embodiment, the invention is directed toward a computer system capable of carrying out the functionality described herein. An example computer system 100 is shown in FIG. 1. The computer system 100 includes one or more processors. A processor can be connected to a communication bus. Various software embodiments are described in terms of this example computer system. After reading this description, it will become apparent to a person skilled in the relevant art how to implement the invention using other computer systems and/or computer architectures.

Computer system 100 also includes a main memory, e.g., 104, preferably random access memory (RAM), and can also include a secondary memory. The secondary memory can include, for example, a hard disk drive and/or a removable storage drive, representing a floppy disk drive, a magnetic tape drive, an optical disk drive, memory card etc. The removable storage drive reads from and/or writes to a removable storage unit in a well-known manner. A removable storage unit includes, but is not limited to, a floppy disk, magnetic tape, optical disk, etc. which is read by and written to by, for example, a removable storage drive. As will be appreciated, the removable storage unit includes a computer usable storage medium having stored therein computer software and/or data.

In alternative embodiments, secondary memory may include other similar means for allowing computer programs or other instructions to be loaded into computer system 100. Such means can include, for example, a removable storage unit and an interface device. Examples of such can include a program cartridge and cartridge interface (such as that found in video game devices), a removable memory chip (such as an EPROM, or PROM) and associated socket, and other removable storage units and interfaces which allow software and data to be transferred from the removable storage unit to computer system 100.

Computer system 100 can also include a communications interface (106). Communications interface allows software and data to be transferred between computer system and external devices. Examples of communications interface can include a modem, a network interface (such as an Ethernet card), a communications port, a PCMCIA slot and card, etc. Software and data transferred via communications interface are in the form of signals which can be electronic, electromagnetic, optical or other signals capable of being received by communications interface. These signals are provided to communications interface via a channel. This channel carries signals and can be implemented using wire or cable, fiber optics, a phone line, a cellular phone link, an RF link and other communications channels.

In this document, the term “electronic storage medium” is used to generally refer to media such as removable storage device, a hard disk installed in hard disk drive, and signals. These computer program products are means for providing software to computer system 100.

Computer programs (also called computer control logic) are stored in main memory and/or secondary memory. Computer programs can also be received via communications interface. Such computer programs, when executed, enable the computer system to perform the features of the present invention as discussed herein. In particular, the computer programs, when executed, enable the processor to perform the features of the present invention. Accordingly, such computer programs represent controllers of computer system 100.

In an embodiment where the invention is implemented using software, the software may be stored in a computer program product and loaded into computer system 100 using removable storage drive, hard drive or communications interface. The control logic (software), when executed by the processor, causes the processor to perform the functions of the invention as described herein.

In another embodiment, the invention is implemented primarily in hardware using, for example, hardware components such as application specific integrated circuits (ASICs). Implementation of the hardware state machine so as to perform the functions described herein will be apparent to persons skilled in the relevant art(s).

In yet another embodiment, the invention is implemented using a combination of both hardware and software. In addition, the data computer system preferably includes a display device, which can be any device for displaying (101) information in a graphical form, a keyboard (107), which can be any device for inputting characters, and a mouse with a button, which can be any device for indicating screen position.

As envisaged by the present invention, the computer system possesses a database. A database may include, but is not limited to, fields of searchable data, author and title information; textual fields that include related annotations or perhaps the full text; contact fields that include all the bibliographic information and text strings for sequence data; catalog data or product information data. In a related aspect, the choice of properties possessed by particular fields may include fields which are searchable and displayable or displayable only.

The present invention may be embodied in a software program residing on a data processing system operating under Unix and/or Windows operating systems. In one embodiment, the software program is written in pearl, C, C++, C# and Java programming languages and uses the relational database management system, as the data storage.

In one embodiment, an electronic commerce site implemented by a computer system is provided where multiple users access the electronic commerce site over a network. The electronic commerce site is capable of presenting product information to the users. The product information may be stored in a database within system so that a vendor may update, add, delete, or otherwise modify product information. The product data may include descriptors, identifiers, or other useful information to the purchasers.

In one embodiment, a data entry function may be provided. The data entry function provides a user or customer the ability to input data. The data entry function accepts customer input and transmits the data over a network. The data entry function may transmit data to a purchasing order function, which receives customer input data and stores, records, executes, or otherwise processes the received data. The purchasing order function, as part of a computer system, processes product order information from the data function, may execute an order command, provide confirmation to the customer of his/her order, or search for product based on customer input. The processes of the purchase order function are not intended to be limited in any way and primarily functions or provides capabilities known to those skilled in the art of electronic commerce systems.

The electronic commerce system may also provided a search function. The search function matches user input to a vendor's database files or catalog. The search function capabilities of the system may be varied, and additional embodiments are described below. Generally, the search function allows a user to search for a variety of information including product information, pricing information, availability, order history, and any number of other commonly used search parameters. Additionally, in certain contexts the search function may provide for advanced search capabilities. Basic and advanced search functions contemplated by the present invention include those disclosed in U.S. Patent Application Publication No. 2005/0240352 (application Ser. No. 10/830,074) and U.S. Patent Application Publication No. 2006/0100788 (application Ser. No. 11/112,933), which are incorporated herein by reference in their entireties.

In certain aspects of the present invention, methods for selling product to a group through an electronic commerce website are provided. The method includes: providing an electronic commerce system to a customer group, in which the customer group that includes at least a first customer and a second customer, and in which the electronic commerce system includes a shared shopping cart to which members of the customer group have access; allowing the first customer to direct one or more products to the shared shopping cart; and allowing a second customer to activate a purchasing function to purchase one or more products in the shared shopping cart, by which means a product is sold to a customer group. The customer group directly or indirectly provides consideration, such as monetary consideration to purchase the product.

In this embodiment, the computer system provides an electronic commerce system with all the functionalities and capabilities described herein. Additionally, the electronic commerce system provides a shopping cart to which multiple users may have access. In this embodiment, the shopping cart is identified with a unique identifier. The unique identifier may be anything that distinguishes one shopping cart from another and that is capable of being associated with more than one user.

Accordingly, a user may (e.g., a customer that is a member of a customer group) open an account in an electronic commerce system and be provided with a unique log on. A shopping cart may be created that is associated with a unique identifier. The unique identifier of the shopping cart is then associated with the user's log on information and the information is stored in a database or memory of the system. In alternative embodiments, users may be associated with a uniquely identified shopping cart by other means than the user's log on information. In such embodiments, any type of information or data may be used as log on information, including account codes, email address, name, address, entity's name, or other information such that a user entering an electronic commerce system may be provided access to a uniquely identified shopping cart. Two or more types of identifiers may be used to associate a user with a uniquely identified shopping cart (e.g., logon plus personal identification number (PIN), user name plus password, etc.). As used herein the user's “logon” will be used to mean the information used to identify a user and allow the user access to the electronic commerce system website.

Upon entry into the electronic commerce website, the system recognizes the user or customer and provides information to the customer according to their association with a uniquely identified shopping cart. The information provided can be any information including account information, past product order information, account activity information, or user privilege information. Once logged on, the customer may then utilize various features of the electronic commerce website to search for one or more products or other information, order one or more products, manage product orders, or manage the account associated with the shopping cart.

In some embodiments, more than one user having a unique user logon may be associated with a uniquely identified shopping cart. In this embodiment, multiple users (customers, for example, members of a customer group) with independent logons have access to a single shopping cart. Each user is then able to perform activities that become associated with the uniquely identified shopping cart. These activities include searching for product or other information, ordering product, managing product orders, or managing the account associated with the uniquely identified shopping cart.

In exemplary embodiments, an electronic commerce system provides more than one user with access to a common shopping cart. The electronic commerce system is capable of displaying product information and other information to each user associated with the shared shopping cart. Thus, if a first user were to submit an order for a particular product, the information could be displayed to other users. In this scenario, each user may be able to see product order information from other users. This allows each user to tailor his or her order purchases when applicable. Additionally, each user may be allowed to modify quantity within the shopping cart. Thus, if a first user orders an item, the second user upon seeing the item ordered may adjust the quantity selected to accommodate the second user's purchasing preferences. As one of skill in the art would understand, the transparent product order information can be used by multiple users to make informed purchasing decisions.

The electronic commerce system with a shared shopping cart may provide additional functionalities. For example, in one embodiment the system may allow users to enter requests for products. In this example, a first user may input a request into the system requesting a particular type of product. The system would then display that information to other users associated with the shopping cart and allow the other users to make purchasing decisions after taking into account the first user's request.

In alternative embodiments, a first user may input comments regarding products or other information. Other users associated with the shopping cart may then make purchasing decisions or take other actions taking into account the first user's comments. In alternative embodiments, the system is capable of associating a user's product order with the name or other identifying information for that particular user. Accordingly, as other users enter the system, the system may display product order information, requests, or comments and identify to the other user the first user's identity.

In one embodiment, a user associated with an electronic commerce system providing multiple users with access to a shared shopping cart is assigned or associated with privileges. In this example, privileges are permissions that specify the type, frequency, degree, and/or scope of actions a user may take with respect to the features of a particular system. For example, a first user may be designated as the account manager for a shared shopping cart. In this example, the first user may have certain privileges associated with the first user including but not limited to display privileges (whether a user will be entitled to see something), product placing privileges (whether a user will be entitled to place product orders), product order privileges (whether a user will be entitled to submit product orders), product order deletion privileges (whether a user will be entitled to delete placed orders), product order quantity modification privileges (whether a user will be able to modify placed orders), end user correlation privileges (whether a user will be able to correlate a second user to a shopping cart), and account management privileges (whether a user will be able to modify account settings including shipping and billing settings).

Thus, a first user with correlation privileges may invite or otherwise designate a second user with the shared shopping cart. Furthermore, upon correlating a user to the shared shopping cart, the first user with end user correlation privileges can further specify the privileges associated with the second user, including whether the second user will have end user correlation privileges.

In one embodiment, a user is designated as the account manager and is associated with manager privileges. In this embodiment, the user may be responsible for making final determinations concerning whether product orders made by other users to the shared shopping cart are placed with the vendor and may be the only user of the shared shopping cart with privileges to access the system's purchasing order function. Additionally, the account manager may make final decisions concerning when orders are placed with the vendor or the frequency at which orders are placed. Also the account manager may be responsible for specifying any number of other parameters associated with the shared shopping cart including billing information, billing options, shipping location, delivery options, reporting options, and/or modification options. Manager privileges may also include, for example, the ability to track product orders that have been placed through the electronic commerce system. Manager privileges may include any number of different privileges including all, some, or at least one of the aforementioned user privileges.

In one embodiment, the electronic commerce system provides search functions to one or more users. In this embodiment, the search function provided by the system provides the user with an ability to search for product offered by the merchant. In this embodiment, a user may conduct searches by any number of means, including but not limited to by textual input, ID number, product key, SKU number, or other means. The user may similarly search for recommended or previously purchased items. Furthermore, the user may search for information concerning a protocol, application, or workflow.

The system provides the user with information concerning the search request that may include links to purchase product. In returning results, the system may provide more than one product option. Furthermore, the system may return results that include previously purchased items or recommended items. The results may be ranked according to preferences specified by the user, or alternatively, by the vendor. The results may list, for example, product information concerning previously purchased products that match the search request first followed by other product information, such as related products and/or recommended products. In one embodiment, the search function provides advanced search functionalities, including but not limited to keyword searching, Boolean text searching, identification number searching, catalog number searching, and/or characteristic searches. In one embodiment, the search functionalities may be further specialized to include advanced search functions for a particular industry and their related products. Examples of such specialized search capabilities are disclosed in U.S. Patent Application Publication No. 2005/0240352 and U.S. Patent Application Publication No. 2006/0100788, each of which is incorporated herein by reference in its entirety.

In one embodiment, the searches performed by a first user may be associated with the shared shopping cart. Accordingly, the results displayed may be displayed to other users associated with the shopping cart. In one embodiment, the account manager may be responsible for ordering products from search results, whether by agreement between the users or by privilege restrictions. In one embodiment, one or both of the search query and/or search result is stored by the system for later viewing and product ordering. In alternative embodiments, one or both of the search query and/or search result may be viewed by other users, including the manager, just the manager, just the user who conducted the search, or users with search review privileges.

As mentioned previously, the electronic commerce system may provide functionalities for entering requests or comments. For example, in one embodiment the electronic commerce provides a module that may be part of the data entry function or separate therefrom, whereby a user may enter input, such as a request or comment. The first user's input may then be seen by other users of the shared shopping cart.

By default, a system may be designed so that all users associated with the shopping cart may see requests. Alternatively, the account manager may be the only user with permission to see user input. In one example, a request for a particular type of product may be inputted by a user. Accordingly, a second user, for example an account manager, may then use the request to place a product order or modify an existing product order.

In one embodiment, the account manager upon receiving a request for a particular type of product, may conduct a search using the search function to identify potential products. The search function may then return results including product previously ordered by the first user. The account manager may then, if appropriate, place an order for the previously ordered product by transmitting product order transaction data to the purchasing function. In cases where the account manager or other user cannot determine the product desired by the first user, the account manager or other user can input text into the data entry function seeking clarification and direct that text to the first user or all users of the system. The account manager or other user may also provide the first user with product options, or otherwise provide the first user with limited ordering privileges so that the first user may order product themselves, if appropriate.

In another embodiment, a second user (that may be an account manager) may provide search results to a first user who submitted the request. Alternatively, additional users may themselves recommend products to the requesting party. In one embodiment, the shopping cart provides an area for user input wherein one user may recommend a product and include textual annotations, such as product usage information or a second user's product performance determination. As one of skill in the art would understand, any number of different implementations of the various features and functionalities described herein can be used by the users of a shared shopping cart.

Another feature contemplated by the present invention relates to reporting features. In one embodiment, the electronic commerce system has reporting features that allow users to view information concerning the shared shopping cart. In one embodiment, only users associated with reporting privileges may conduct, order, execute or run the reporting features. In alternative embodiments, reporting features may be given to one, more than one, or all users associated with the shared shopping cart. In alternative embodiments, reporting privileges may further be subdivided into different privileges such that some users may have limited reporting privileges.

In one embodiment, a reporting function may be provided as part of the data entry function or product ordering function. Alternatively, the reporting function may be a separate module that is part of the system. In one embodiment, the reporting function may provide a user with information concerning product ordering transaction data associated with a shared shopping cart. The reporting function may be capable of parsing and displaying data according to various parameters. Such parameters may include, but are not limited to, date, time, users of the shared shopping cart, product, product type, product characteristics, amount, billing codes or other reference codes, upcoming orders, pending orders, past orders, or other parameters. Accordingly, a user with reporting privileges may input requests and have the reporting function return results in various forms to the user. The returned report may organize, display, collect, or otherwise provide the user with useful information concerning the shared shopping cart.

In one embodiment, the system may provide a user with access to a traditional shopping cart and a shared shopping cart. As used herein, traditional shopping cart refers to a shopping cart to which only one user is provided access. In such cases, the shared shopping cart may be an optional ordering method available to the user in an electronic commerce system. The shared shopping cart may be referred to by any number of distinguishing means, including for example, a virtual whiteboard. The user may decide in some instances to enter requests, comments, or product orders into a virtual whiteboard (shared shopping cart), or alternatively decide to enter product orders through a traditional shopping cart. Accordingly, in one aspect of the present invention, an electronic commerce system is provided in which users may browse, search, or specify product prior to determining whether the user input will be associated with a traditional shopping cart or a virtual whiteboard.

Alone or in combination, the above described features provide methods of processing product orders, generating revenue, and/or selling product that is particularly suited for vending product to individuals in group environments. Of particular benefit to providing such a service is that one vendor or merchant can increase sales by providing one-stop shopping for the products necessary to a particular organization. A “group environment” can be a group of individuals working in a common physical location, or individuals working at different sites belonging to the same company, organization, or institution, or individuals at different institutions working collaboratively or under an agreement, such as a contract agreement or grant. All of these groups of individuals can include customer groups, which may comprise some or all of the individuals of a group working collaboratively or under an agreement. For example, in the area of biological research, different members of a university or research institute-based laboratory, or research and development or product development department or division of a company can have access to a shared shopping cart (virtual whiteboard). Individuals working in different laboratories of the same or different institutions or companies that have collaborative arrangements can also have access to a common shopping cart.

In some embodiments, the electronic commerce system includes a range of products and services for purchase that are used in different areas of a field of research, such as, for example, different areas of life sciences research, such that products and services can be provided by the vendor (or provider of the electronic commerce system) to multiple customers within a laboratory, department, institute, or enterprise that form a customer group, although individual customers may work in different research areas and use different technologies. For example, a vendor in the life sciences business that provides or uses an electronic commerce system having a customer group/virtual whiteboard capability can provide products or services for focus areas such as but not limited to: drug discovery, proteomics, gene regulation, developmental sciences, molecular neuroscience, signal transduction, immunology, cell death/apoptosis, cell migration, and use such varied technologies as flow cytometry, in situ hybridization, cell culture, immunoassays, mass spectrometry, polymerase chain reaction, immunohistochemistry, electrophoresis, transgenic animals, protein purification, biochemical assays, cellular assays, nucleic acid purification, organelle isolation, etc.

In another aspect of the invention, methods for selling research products associated with workflows, applications, and protocols that are provided on the vendor's website are provided. In some embodiments, the method includes: providing an electronic commerce system to a customer, in which the electronic commerce system provides an electronic representation that includes a protocol, in which the electronic representation includes a means for purchasing a product used in at least one step of the protocol. The electronic representation can be, for example, a display, such as a web page that can be visually displayed on a monitor or screen, and the means for purchasing a product can be by providing on the same display a designation, name, or information pertaining to a product having one or more links that directly or indirectly access a purchasing function.

A designation, name, or information pertaining to a product includes, without limitation, any of: the product name, including any alternate names, chemical names, or common names; the product size, weight, quantity, concentration, composition, purchase unit quantity or volume, activity, absorbance or fluorescence wavelengths, color, grade, purity, common uses, source, catalog number, product identification number, SKU, price, regulatory information, recommended or proscribed use, precautions or warnings, and designations, names, or identification numbers of alternate products or related products.

In some embodiments, the method includes: providing an electronic commerce system to a customer, in which the electronic commerce system includes a database of searchable protocols that can be displayed to the customer such information about one or more products associated with a protocol is either juxtaposed with the protocol on the same display, or electronically linked to the protocol, and in which the product information is directly or indirectly linked to a purchase function on the electronic commerce system. In these methods, one or more products are sold to a customer who accesses a protocol and activates a purchasing function for a product associated with the protocol. The purchasing function can generate an order for the product or products and a bill or direct transfer of payment of consideration, such as monetary consideration to the provider of the electronic commerce system. The purchasing function can optionally use a shopping cart, and can optionally used a shared shopping cart, as described herein.

In conjunction with an electronic commerce system, a data entry function and search function provide users the ability to search and locate workflows, applications, protocols, biomodules, kits, matched reagent sets, and other product groups. In the case of a search request, such queries may relate to a particular type of goal desired, whether it is an amplification protocol for biological research, or other protocols, such as for assays, analyses, detections, separations, genetic or biochemical manipulations, or preparations or modifications of cells or biomolecules.

In one embodiment, the protocol provided by the electronic commerce system may be a well-established protocol. In some embodiments, the protocol may be a well-established scientific protocol, a well-established biological protocol, a well-established biological research protocol, or a well-established biological reagent research protocol. As used herein, “well-established” refers to a protocol, process, application, workflow, or other series of steps that has been used by a community and delivers consistent, repeatable, validated, or accurate results. A well-established protocol, in some instances, is a protocol that has been published in a book, in certain embodiments a hard-cover book that has sold more than 100 copies, or more than 500 copies, or more than 1,000 copies, or more than 5,000 copies. In other instances a well-established protocol is a protocol that has been published in more than 10 scientific publications or peer reviewed journals or peer reviewed articles. In alternative instances, a well-established protocol has been published in more than 100, or more than 200, or more than 500 scientific publications or peer reviewed journals or peer reviewed articles.

In one embodiment of the present invention, a vendor with an electronic commerce system (for example, a website used for commerce) licenses a well-established protocol from a third party. In some embodiments, the third party is a book publisher, a non-commercial entity, or a commercial entity. In one embodiment of the present invention the protocol pertains to scientific research. In other embodiments, the protocol relates to biological research or research that includes the use of biological reagents or materials, such as cells, cell fractions, and biomolecules.

In some embodiments of the invention, a protocol provided on the vendor's electronic commerce system (e.g., website) is a protocol that has been published outside the vendor's electronic commerce website, such as published by a commercial entity other than the provider of the electronic commerce system that displays the protocol, and other than the provider of products that are associated with the protocol on the electronic commerce system, in which the published protocol (or a book, journal, article, or other medium that includes the protocol) is copyrighted. As used herein, a copyrighted book, journal, or other printed publication is a book, journal, or other printed publication having a registered copyright, in which the copyright has been registered with a government agency. In some embodiments, a protocol provided on the vendor's electronic commerce system is a published protocol in which the text is copyrighted, and the copyrighted protocol is not available to an individual such as a customer who does not purchase the protocol (or a book, manual, or journal that includes the protocol) other than through the vendor's electronic website, which allows the user to view the protocol without payment or remuneration to the vendor or the publisher of the protocol. Thus, the published, copyrighted protocol provided on the electronic commerce website in a format in which products useful in at least one step of the protocol, is not freely available to a member of the public other than through accessing the electronic commerce system.

Purchase of a book, manual or journal includes purchase that occurs online, including online subscription services that may provide entire books or manuals, sections or chapters of books or manuals, journal articles or sections (e.g., appendices), or subscriptions to journals for a fee. Thus, in some exemplary embodiments of the invention, the electronic commerce system provides a customer with free access to a protocol to which the publisher of the protocol does not otherwise provide free access. In preferred embodiments, the protocol has been published by a commercial entity in a book or journal, preferably a book, such as a hardcover or softcover book which is or has been available for purchase, and has a copyright registered with a government agency.

In preferred embodiments, the copyrighted book or other printed work that includes the protocol is not a product manual. In exemplary embodiments, the copyrighted book or printed work has been published and printed by a commercial entity that does not provide products for use in steps of the protocol.

In some embodiments, the present invention provides a method for generating revenue by providing a viewable protocol on an electronic commerce system, in which the protocol is viewable by the customer without monetary consideration by the customer either to the provider of the electronic commerce system or to a publisher or copyright owner of the protocol, in which the protocol is associated with a purchase function for purchasing one or more products that can be used in one or more steps of the protocol. In these methods, a customer directly or indirectly accesses a purchase function for one or more products that are designated on the display that includes the protocol. The customer activates a purchase function to buy a product that can be used in one or more steps of the protocol from the provider of the electronic commerce system, by which means revenue is generated by the electronic commerce system. The provider of the electronic commerce system can be the provider of the product, or can be a separate entity that provides a commercial link between the product provider and customer, and receives a portion of the purchase price of the product as revenue. The owner of the copyright of the protocol can be the same as or different from the electronic commerce provider and/or product provider. In some embodiments, the owner of the copyright of the protocol is a separate entity from the product provider, and the copyright owner receives consideration (typically monetary consideration) for purchase of a product designated in, linked to, or juxtaposed with the protocol on the electronic commerce site, by a user who accesses the viewable protocol.

In exemplary embodiments, the published copyrighted protocol provided on the electronic commerce system free of charge is provided to the user on a display that also includes a list of products useful in performing the steps of the protocol, in which the products are available from the provider of the electronic commerce system, and a purchasing function for purchasing the listed products can be electronically accessed by the user from the display that includes the protocol and the list of relevant products.

In some embodiments, terms of use for the copyrighted protocols can be available on the electronic commerce system site. Such terms can include, for example, prohibition of one or more of copying, printing, downloading, modifying, diassembling, derivatizing, disseminating, or selling the copyrighted protocols. In some embodiments, the electronic commerce system does not provide a means for formatting the displayed copyrighted protocols to be compatible with printing, and does not provide a print function for directly printing the protocols, for example, though use of a GUI print icon provided by the commerce system.

The electronic commerce system in exemplary embodiments provides a plurality of protocols that are preferably provided in a common searchable database or format. In exemplary embodiments, one or more of the protocols is an established protocol. In some exemplary embodiments, one or more of the protocols is a published, copyrighted protocol. In some exemplary embodiments, the one or more published, copyrighted protocols has been published or copyrighted by an entity other than the commercial electronic system provider or product vendor.

In some exemplary embodiments, the electronic commerce system provides an extensive set of protocols that appear together in a published and copyrighted book, or that appear in a set or collection of books (e.g., book volumes of a series), or that appear in a series of articles that are published in a journal, which can be a traditional print journal, an electronic journal, or a journal that is provided both as a traditional print journal and is also published electronically (e.g., an “on-line” journal). In some exemplary embodiments, the electronic commerce system provides all of the set of protocols that appear together in a published and copyrighted book, or that appear in a set or collection of books (e.g., book volumes of a series), or that appear in a series of articles that are published in a journal, which can be a traditional print journal, an electronic journal, or a journal that is provided both as a traditional print journal and is also published on line.

The copyrighted protocols provided by the electronic commerce system preferably include protocols from more than one research technology or methodology, and preferably from more than one biology research technology or methodology, where a biology research methodology or technology can be, for example and without limitation, cloning (recombinant DNA technology), transfection, cell culture, electrophoresis, mass spectrometry, nucleic acid isolation and analysis, nucleic acid amplification, protein expression and analysis, flow cytometry, immunobiology, cellular assays and analysis, gene regulation, signal transduction, cellular and developmental neuroscience, or molecular neuroscience. The protocols provided on an electronic commerce website can include protocols in two or more, three or more, four or more, five or more, six or more, seven or more, or eight or more of these methodologies or technologies.

An electronic commerce system can include protocols that are not copyrighted and protocols that are copyrighted, and copyrighted and noncopyrighted protocols can be searchable together (e.g., they can be provided in the same searchable database), and, optionally, provided in a common index of protocols. Preferably, the copyrighted and noncopyrighted protocols have separate designations indicating the source of the protocols to the user. Both copyrighted and noncopyrighted protocols can have associated product designations or information that is directly or indirectly linked to a purchasing function.

In one embodiment of the present invention, the electronic commerce system displays information concerning a well-established protocol, such as a copyrighted protocol not available outside of the electronic commerce system except through purchase of a book, manual, or journal that includes the protocol, to a user of the system. The display may be a GUI or other display format that provides an electronic representation of textual information concerning one or more protocols in response to a request by a user. The displayed protocol may be as a result of browsing displayed protocols by categorical indices, descriptive links, or as a result of a search request.

The user may then select a protocol desired and review the steps attendant to the protocol. The electronic commerce system, through an electronic representation of the protocol may include links to one or more products necessary to perform the steps of the protocol, or useful in performing the steps of the protocol. Where a protocol requires more than one product, the electronic commerce system may display each product necessary for the different steps of the protocol.

The electronic commerce system may electronically represent product information in proximity to the protocol. Thus, for example, where a series of steps is provided, the electronic commerce system may represent links to the product necessary to execute a particular step to the left or right of the protocol in a separate location of the GUI. The products can optionally be listed to the right or left of the protocol in proximity to steps of the protocol in which they are used. Alternatively, all products necessary for the entire protocol may be listed at the top or bottom of the protocol. One of skill in the art would understand that links to the required products could be moved within the display to accommodate different formatting and display preferences.

A list of products juxtaposed with the protocol on the same display page can include the name(s) and product numbers of the products useful in performing the steps of the protocol, and can optionally include other information, and can also include links, including but not limited hyperlinks embedded in a brief description of the product, that allow the user to access further description of the product, which may include further links or hyperlinks to other products or information, and/or a purchasing function for purchasing the product.

In one embodiment, a list of products is provided above, below, or to the right or left of a protocol, such as a copyrighted protocol, displayed on the electronic commerce system, and the protocol does not include hyperlinks to products. The protocol may include hyperlinks to other information, such as, for example, articles, commentary, footnotes, other protocols, tables, or figures. Such other information can optionally be part of the same copyrighted work (e.g., book or manual) that the copyrighted protocol that includes the hyperlinks. In some embodiments, the hyperlinks may link to information on an internet site that is not part of the electronic commerce system, and can be, for example, a public database.

In one embodiment, hyperlinks within the protocol link to data entering functions for placing the product into a shopping cart. The links or data entering function may be provided with icons to place a product into a shopping cart without displaying additional information in the event that a user does not wish to leave the page displaying the protocol.

In one embodiment, the electronic commerce system displays at least one of the products necessary to accomplish a step of the protocol. In other embodiments, the electronic commerce system displays more than one, at least half, or substantially all products necessary to accomplish the steps of the protocol. The precise number entailed by substantially all products necessary to accomplish the steps of a protocol varies and relates to the particulars of the protocol. Thus, for example, where a protocol requires one re-usable component or product and four perishable or one-time use components or products, a vendor may decide to provide links to the four perishable components. In this example, the vendor may determine that its customers typically already have the reusable component and thus, it is not included in the product list necessary to accomplish the protocol.

In other embodiments, substantially all product associated with a protocol includes where a vendors provides links to one type of product used in the protocol. Thus, where a protocol uses equipment and reagents, a vendor may provide links to all reagents needed to execute the steps of the protocol. In this example, a vendor has provided substantially all products necessary to execute the steps of a protocol.

In other embodiments, substantially all product associated with a protocol includes where a vendors provides links to products that are not commonly found in the particular setting and may decide not to provide every single product of a product type for the protocol. For example, in biological reagent research protocols, the steps may require a number of reagents, one of which may include deionized, purified water. According to the precepts of the present invention, a vendor may omit said reagent from the product information display as it is a commonly used item, which the vendor has determined is not typically needed by customers executing the protocol of interest. Another example includes where a vendor may decide not to include in its product offerings items such as commonly stocked salts, acids, bases, tubes, pipettes, incubators, minerals, oils, balances, or other common laboratory reagents or equipment. One method by which vendors can determine which products to provide with respect to a particular protocol involves determining which products are specific to the particular protocol being displayed. Thus, with respect to biological research reagent protocols, a protocol pertaining to acid precipitation of protein samples may provide links to 4,4′-dicarboxy-2,2′-biquinoline, disodium salt but not to sodium hydroxide or water. In this example, the vendor has determined that the salt is not a commonly stocked item and specific to the protein precipitation protocol. Alternatively in protocols for the amplification of DNA by the polymerase chain reaction protocol, a vendor may offer primers as substantially all products necessary to complete the protocol but not additional reagents as they may be common to the laboratory setting.

In some embodiments, individual product designations or names have separate links that directly or indirectly access a purchasing function, and in preferred embodiments, further product information, including other products that are related in function, are part of a matched reagent set, or can be used in the same workflow or biomodule.

In one embodiment, the electronic commerce system provides kits or product groupings for performing the selected protocols. In this embodiment, in addition to the individual product listings for products necessary to complete the steps of a protocol, a vendor may provide separate links to product groups, matched reagent sets, kits, or biomodules. A product group may be a single link that when actuated by the user either displays or places in a customers' shopping cart substantially all of the individual products associated with the protocol. A kit similarly provides a single link to a grouping of products associated with the protocol. A product grouping may differ from a kit in that the product grouping is a link of individual components or products, whereas a kit is a pre-prepared grouping of components or products. Accordingly, from within the shopping cart, a user may modify a product grouping by removing or adding individual products from the product groupings. Kits typically come pre-prepared and their components are generally not modifiable.

In one embodiment, the electronic commerce system may also provide links to equivalent protocols. In this embodiment of the present invention, vendors may provide protocols and attendant product offerings within the electronic representation of the protocol. Thus a user may be provided with a well-established protocol and the steps thereto. Within that display, the user may be provided a link to an equivalent protocol that achieves generally the same results as the well-established protocol. The link may be to a description of the equivalent protocol, or its attendant biomodule, kit, or product group. In some embodiments, the equivalent protocol may provide better or superior results. In other embodiments, the equivalent protocol may provide more efficient execution, less down-time, less preparation, or any number of advantages over the well-established protocol.

In one embodiment, the products, product groups, matched reagent sets, kits, biomodules, equivalent protocols and equivalent protocol products have been validated. Accordingly, a user will know that the selected products, product groupings, etc. are compatible with each other within the selected protocol or equivalent protocol and that the set of components and products have been tested in combination and been shown to cooperatively perform all of the steps of the application or protocol using an internal control, or its derivative, wherein the derivative is derived from the steps of the application or protocol itself.

As one of skill in the art would understand, the electronic commerce system that provides product designations, lists, or links on the same display as protocols may provide users with the option of using a shared shopping cart or not. Accordingly, when selecting protocols and the attendant products, users may be offered the ability to place ordered products, kits, biomodules, and equivalent protocol products into a shared shopping cart or a private shopping cart.

While the methods of the invention are not limited to such environments, the methods find particular usefulness when groups of individuals share products. Alternatively, the methods of the present invention can be used as a product management system. For example, a research institution may be comprised of different organizational centers, departments, or other groupings such as laboratories. Each laboratory or other grouping may be responsible for research in one particular area. However, each laboratory may use similar products in conducting research. Accordingly, the system of the present invention may allow an account manager to see product requests from each laboratory, even where the individuals within each laboratory only sees product requests and other input from their laboratory. The account manager can then make purchasing decisions in bulk, or make purchasing decisions with the needs and requests of each individual group in mind. Alternatively, each laboratory may be able to see the requests of other laboratories within the research institution. This information can be used by individuals within the laboratory to make product order decisions. For example, where one individual notices that one laboratory is conducting amplification experiments, the individual may adjust his research to coincide with those activities and conduct similar experiments at a different time than originally planned. In this way, the research institutions may be able to place a large product order to realize purchasing efficiencies.

In alternative embodiments, the methods presented herein can be used in conjunction with on-site storage facilities. As discussed previously, and particularly with respect to research institutions, vendors often provide entities with product at an on-site facility. The system and methods of the present can be used in conjunction with such on-site product facilities. Accordingly, product orders from various laboratories within the research institutions can first be matched against product from the on-site storage facility. Where present, product orders can be filled from said facilities rather than being ordered from the vendor directly. Furthermore, the vendor may have access to product order requests filled from the on-site storage facility and may re-stock the on-site storage facility accordingly. Moreover, by being able to track product orders (whether filled from the on-site storage facility or otherwise) a vendor may adjust the type and quantity of products stocked in the on-site storage facility. In this way, the methods and system provided herein can be used as a product management tool by the vendor to make determinations about product offerings. As one of skill in the art would understand, the methods and systems of the present invention find particular use in settings where groups of individuals share, collaborate, or use similar products.

### EXAMPLES

The examples set forth below illustrate but do not limit the invention.

### Example 1

### Virtual Whiteboard

One example of a collaborative setting is a laboratory, whether commercial or educational. In such environments, groups of scientists often work together on projects or in proximity to each other, whether physically, as in adjacent laboratories, or organizationally, such as in a same or a similar organizational department, or financially, as in the case of scientists collaborating under the same funding source. The methods of the present invention are particularly well suited to such environments and organizational settings.

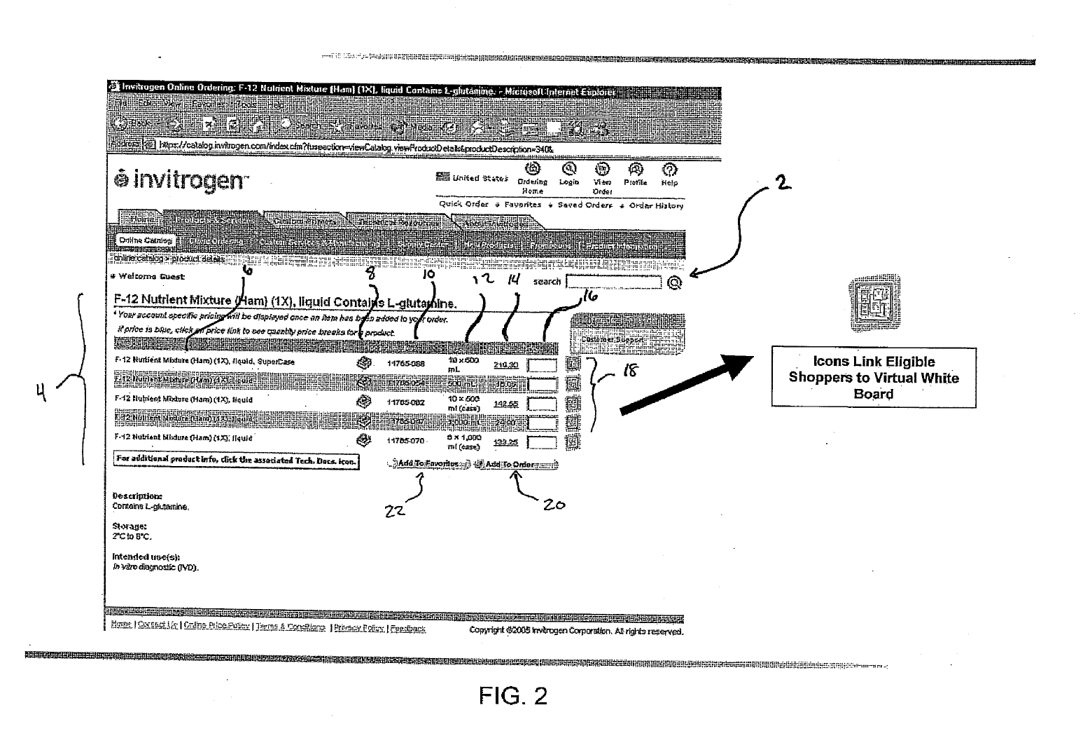

The methods disclosed herein may be used in conjunction with known and existing electronic commerce sites. For example, the methods disclosed herein may be provided as part of a supply center management system for biological reagents as disclosed in U.S. Patent Application No. 2005/0240352, the entirety of which is disclosed herein by reference. As seen if FIG. 2, an exemplary screenshot of a supply center management system as part of an electronic commerce system is shown.

As seen in FIG. 2, the supply center management system may contain various features for the identification, searching, and procurement of relevant biological products and data. Once identified, biological product transaction data may be placed into a shared shopping cart or virtual whiteboard by the user. As seen in FIG. 2, the supply center management system may contain a search function 2 whereby a user from a biological institution may enter input to search for product. Once executed, the system may deliver search results 4 and display them to the biological researcher. The search results may include, for example, product name 6, a link to additional technical product information 8, catalog no. 10, size 12, and list price 14. Additionally, the display includes a quantity field 16 in which the user may enter the quantity of the product desired.

After selecting the appropriate quantity (or alternatively the system may default to certain values if not selected), the user may then place an order for the desired product by clicking on the shared shopping cart icon 18. In alternative arrangements, an ordering link 20 is provided that adds a product to the shared shopping cart. In those instances, the shared shopping cart icon may direct users to a new page displaying shared shopping cart information without ordering an product.

A “favorite” link 22 may be provided wherein a user can identify a product as one frequently ordered or preferred by the user. The system may then associate that item to a user's profile or particular search request. In some embodiments, a user's “favorite” information can be displayed to other users of the shared shopping cart or may be associated with future searches for product by that user or other users.

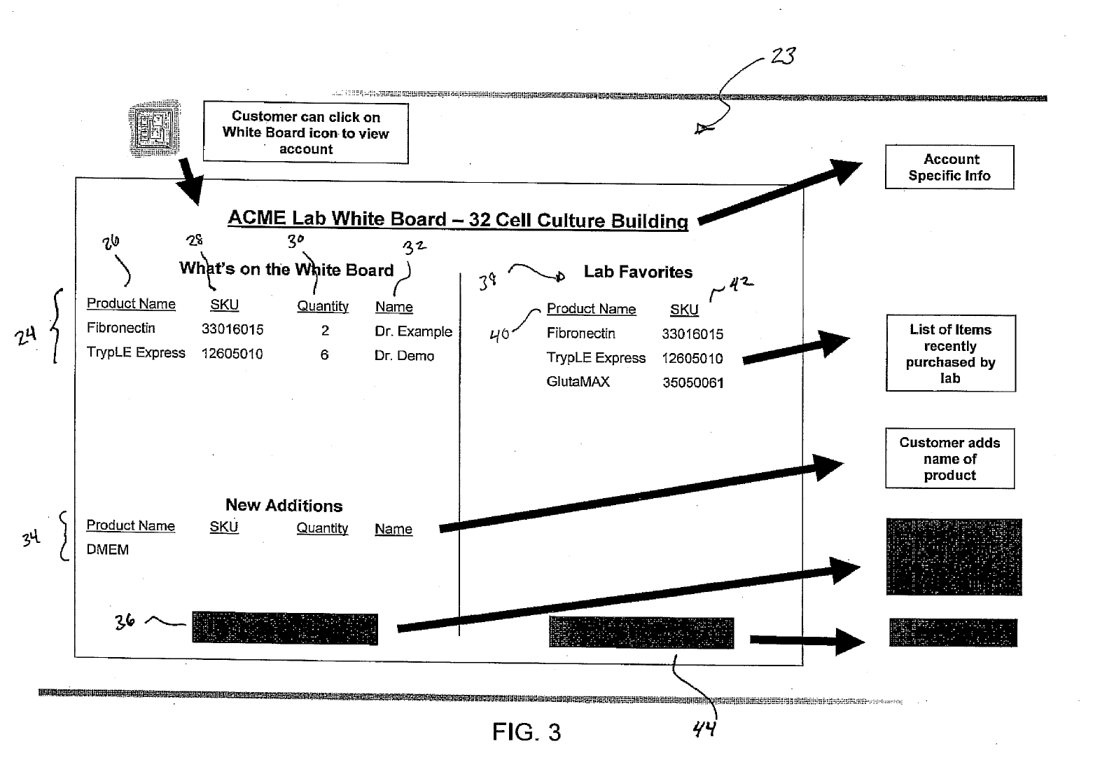

As seen in FIG. 3, a virtual whiteboard 23 or shared shopping cart is shown. In this example, the virtual whiteboard displays currently ordered items 24. The whiteboard may display information concerning the product including but not limited to product name 26, SKU 28, quantity ordered 30, and user name 32. The virtual whiteboard may also have a section for displaying recently added items 34. Recently added items section 34 may display products recently selected from the electronic commerce system prior to adding them to ordered items section 24. This feature allows a user to review the products currently ordered before adding product to ordered items section 24. After review, a user may then add product to ordered items section 24 using link 36. Further sections may include space to display product favorites 38, including product name 40 and SKU 42, and a link to the supply center management system, catalog, or other electronic commerce system 44.

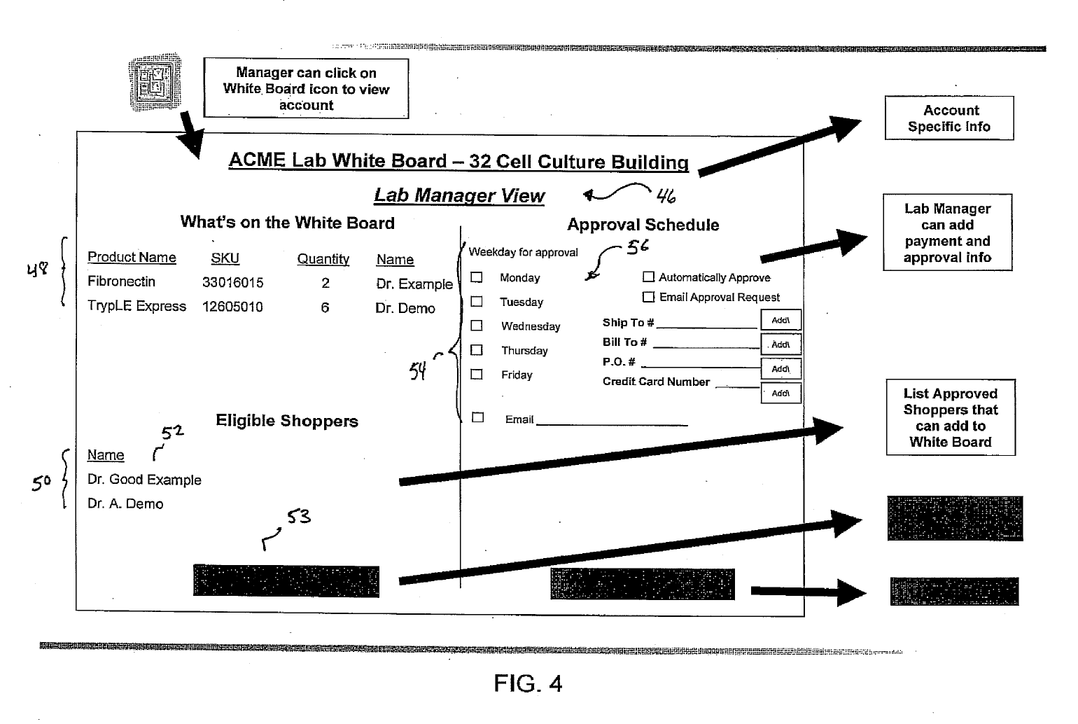

As seen in FIG. 4, alternative views may be provided to particular users. FIG. 4 shows a virtual whiteboard as seen by a user with account management privileges. In this case, the system recognizes the user and displays information concerning the shared shopping cart. As seen in FIG. 4, the whiteboard indicates that a particular view is being displayed by indicating the “lab manager view” 46. The whiteboard contains an ordered items section 48 that lists the various items ordered by the users of the shared shopping cart. The whiteboard also contains an eligible user section 50 that lists the various users eligible to place product orders. The section may include information relating to the user including user name 52. Additionally, the eligible user section 50 may contain link 53 that provides the user with the ability to add additional users. This link may only appear if the user has the appropriate end user correlation privilege.

As seen in FIG. 4, a section of the whiteboard may be reserved for account management in account management section 54. Account management section 54 may provide the account user with control options. The control options may vary depending on the security privileges associated with the particular user. In this example, account management section 54 contains an approval schedule section 56 that allows the account manager to specify account information and product ordering schedules. As seen in FIG. 4, the account manager may select a particular day of the week in which product orders are submitted to the electronic system. Additionally, the account manager may add payment information related to the transaction. A link 58 may be provided that allows the account manager to be notified by email when product transactions have taken place.

Alternative sections may be provided within a whiteboard to allow users to input textual data related to requests or comments. For example, a user may input a request in shorthand for a particular item, such as “need more oligos”. In this case, the data entry function and search functions of the system perform a search that identifies relevant products, including previously purchased products if applicable, and returns the results to the user. The user may then select the biological research reagent product or products from a list. Alternatively, the shared shopping cart feature allows a user to enter a request and have a second user determine the product to order. Where the second user can determine the product desired based on the search results or prior ordering history, the second user may then place an order for the product. If the second user cannot identify the particular product desired, the second user may then provide the first user with a list of items from which to select. The list of items may be the returned search results or a list of items created by the second user or a list of modified search results or both.

In addition to particular product requests, users may input requests for biomodules, workflows, or protocols. The system of the present invention can identify such requests and return appropriate information. For example, a biological researcher may request “reagents for cloning His-2A gene”. As should be understood, the researcher can enter this information in shorthand or as sentences, phrases, or a short sequence of words into the data entry function. Accordingly, the data entry function provides a researcher with the ability to enter product requests or search requests. As such, a request may include things such as “need more oligos for amplifying the human P53 coding sequence” or “we need more 50 ul pipet tips”. The data entry function may then use advanced searching functions to identify particular products. Alternatively, as described previously, a lab manager or other user in charge of managing product orders can use the first user's input to determine the appropriate products to order. If the first user's input is not clear to the second user, the second user may seek clarification and either provide the first user with a list of products returned by the second user's search request, or alternatively seek additional clarification from the first user. The computer system of the present invention contemplates functionalities, whether by software modules or otherwise, that allow users to communicate with each other in shorthand form for the purpose of ordering products.

In this example, the search function may identify the necessary reagents required to perform the requested application and return them to the user. The user could then order reagents associated with the biological application. Alternatively, the user may be able to adjust the quantity and type of biological reagents returned by the search function, if desirable. The search results may also contain biomodules or kits capable of executing the request. Search results may also return equivalent protocols and associated products to perform the request.

As one of skill in the art would understand, where a first user inputs a request related to a biological application or protocol, the request may be transmitted or shown to a second user to make efficient purchasing decisions. The second user may then determine whether to approve the appropriate biological reagent products or may modify the biomodule or kit. Especially as it relates to product groups, matched reagent sets, kits, or biomodules, a second user may review the currently ordered items of a whiteboard and make determinations concerning whether to order the requested biological reagents. This feature is particularly useful in situations where, for example, a request for biological research reagents related to cloning has been made and the returned results include a commonly used biological reagent. A second user (with the appropriate privileges) may determine that the laboratory has sufficient quantities of the biological reagent on hand, or knows that a separate order of the reagent in larger quantities is desirable. Accordingly, the second user may adjust, modify, or otherwise edit the first user's returned product list, including components of the kit, matched reagent set, or biomodule. This feature provides increases flexibility and efficiency of product ordering and processing.

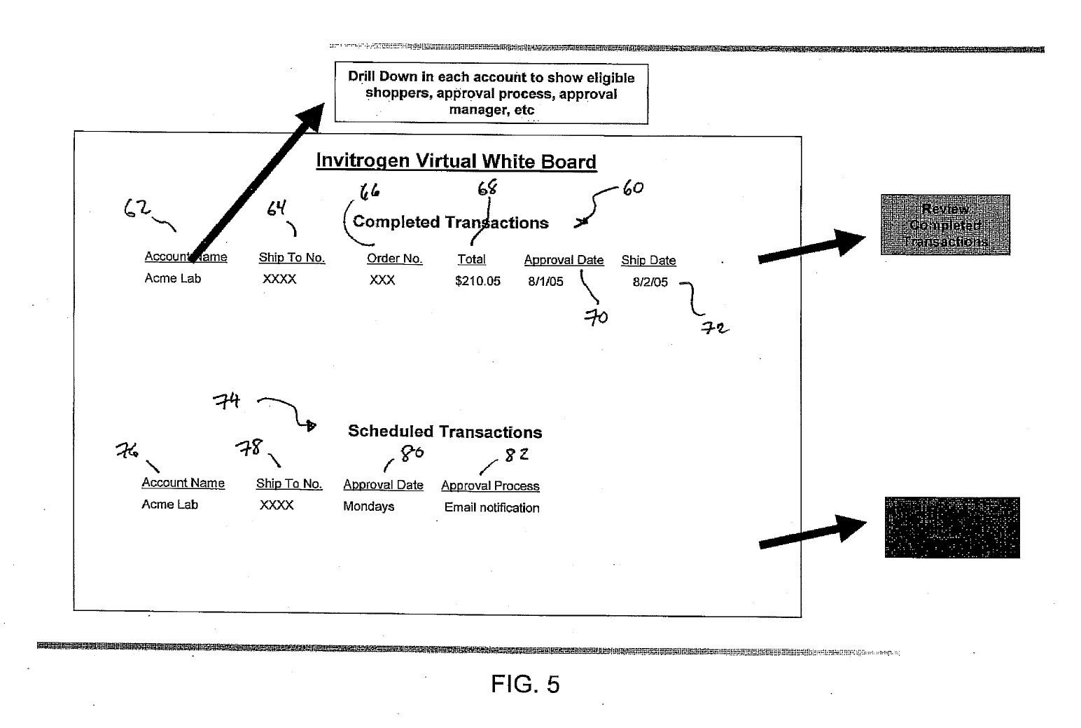

With reference to FIG. 5, the virtual whiteboard may also provide reporting functions. A user with reporting privileges may access the reporting functions. As seen in FIG. 5, the reporting function contains a completed transactions section 60 which lists various data related to completed transactions for the shared shopping cart. Completed transactions sections 60 may list the account name 62, ship address 64, order number 66, order total 68, approval date 70, and ship date 72. As one of skill in the art would understand, various other parameters relating to reporting may be included. As seen in FIG. 5, the virtual whiteboard may also have a scheduled transactions section 74. Scheduled transactions section 74 may display upcoming transactions that have been scheduled by the account manager. Scheduled transactions section 74 may include account name 76, shipping address 78, approval date 80, and approval process field 82. As one of skill in the art would understand, various other parameters relating to reporting may be included.

Additionally, fields within the reporting display may provide access to additional information. For example, account name field 62 may contain a drill down application that provides additional information such as eligible shoppers, approval process, approval manager, or other parameters. The reporting function may also contain saving, exporting, printing, or other archival functions for storing or displaying the reports.

While the present example provides an electronic commerce system with a GUI which may be considered a traditional workstation computer, alternative structure can be employed to practice the disclosed methods. In one alternative example, the data entry function provides the interactive features of the electronic commerce system on a large monitor, much like a whiteboard. In this example, the monitor is capable of receiving user input by traditional devices such as keyboard, mouse, or “stylus” or tablet pens or touch screen technologies. Accordingly, a researcher may enter input by writing on the monitor a request such as “I have run out of BSA reagent—need more”. The monitor might continue to display this request to the user on the monitor until the product is ordered. Similarly, the user may provide other information concerning his/her request such as account information, which is then used by the account manager. Accordingly, the user may enter a request followed by “please charge NIH grant 34522.1”, against which the lab manager can charge said product.

Other monitors part of the system might similarly see the display so that a user may share said product with the first user. A lab manager may decide to order additional reagent for the first user after seeing the display, whether the first user's display or some other display part of the system. In one example, the first user's entry might remain on his/her monitor until the product is ordered. Once ordered by the lab manager, the first user's monitor could display the status of the order, including date order, expected arrival date, quantity ordered, etc. In this manner, the first user will know when his request has been acted upon.

### Example 2

### Protocol Product Reagents for Biological Research

Within the scientific community, and in biological research institutions particularly, protocols are commonly used tools. Book publishers have collected well-established protocols and compiled them for use by the scientific community. One such example of a book containing well-established protocols for the biological research community is Current Protocols in Molecular Biology© 2005 published by John Wiley & Sons, Inc.

As described previously, a number of protocols may be offered by a vendor to customers. The electronic representation of the protocol may include links to products, product groups, kits, biomodules, equivalent protocols and equivalent protocol products as described previously.

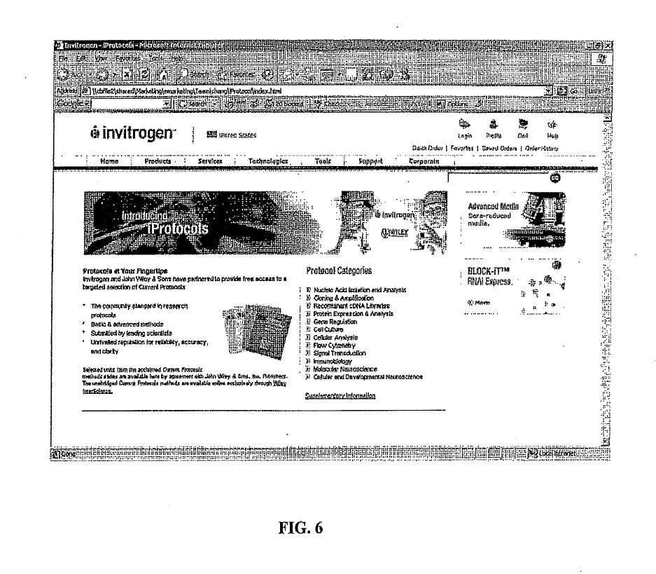

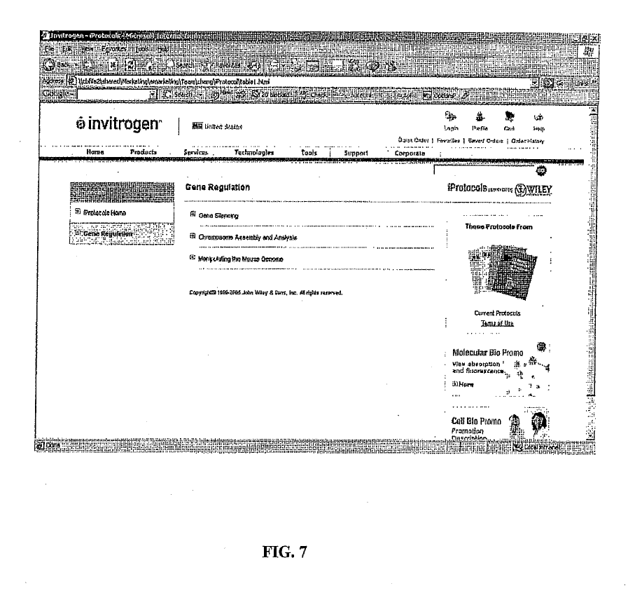

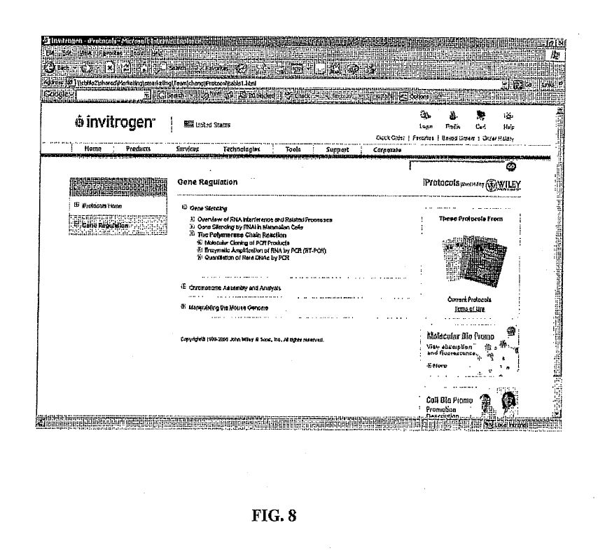

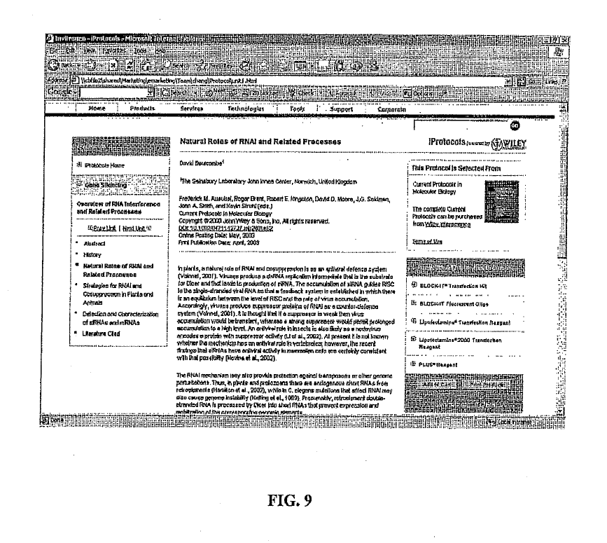

With reference to FIG. 6, a screen shot from an electronic commerce system is shown. As seen in FIG. 6, the electronic commerce system may display a listing, by categories, of well-established protocols. A user may browse available protocols or alternatively may search for protocols. As seen in FIG. 7, if browsing for a protocol, a user may be provided sub-categories. Each sub-category of the electronic commerce system may further be subdivided into additional sub-categories if appropriate. As seen in FIG. 8, additional descriptive links, protocol links, technical summaries and overviews may also be provided. As seen in FIG. 9, additional information may be displayed to the user concerning the particular subject matter of the protocol of interest.

Once selected, the protocol may be displayed to the user. For example, one such protocol may be a protocol for the isolation and expression of recombinant proteins from insects cells, which is reproduced below:

Entry Clones Preparation and Plasmid Isolation:

E. coli cultures of human clones are inoculated into 2 ml deep well culture plates with 900 μl of 2×YT media containing 50 μg/μl Ampicillin and 50 μg/μl carbenicillin and incubated in a 37° C. floor shaker for overnight growth (220 rpm). The next day, plasmids containing hORF clones are isolated by Eppendorfs Perfectprep Plasmid 96 Vac Direct Bind kit (Eppendorf). Plasmid DNA is eluted with 70 μl of Molecular Biology Grade Water. Quality and quantity of DNA are visualized by running 5 μl of isolated plasmid DNA on a 1% E-Gel 96 agarose gel (Invitrogen).

LR Reaction into pDEST 20 Vectors:

The LR reaction is performed in a 10 μl volume in a 96-well PCR plate with the above entry clones and the destination vector pDEST20. 2.5 μl of the following mixture: 100 μl of LR reaction buffer (5× stock, Invitrogen), 50 μl of resuspended pDEST20 DNA (6 μg) and 100 μl of LR clonase (5× stock) is aliquoted into each well of a 96-well PCR plate, and 2.5 μl of the isolated entry clone plasmid is added into each well. The plate is sealed with an aluminum foil cover, spun down at 3000 rpm briefly and incubated at 25° C. overnight.

Transformation of pDEST20 LR into DH10Bac:

40 μl of DH10Bac competent cells are dispensed into each well of the 96-well plate containing the overnight LR mixture. A plate containing the cell mixture is incubated at 4° C. for 15 minutes, and then cells are heat-shocked at 42° C. for 40 seconds. After chilling, 120 μl of LB media are added to each well and the plate is incubated at 37° C. for 5 hours without shaking. At the end of the 5 hr incubation, 50 μl of cells are diluted into 500 μl of LB media containing Gentamycin (7 μg/μl), Kanamycin (50 μg/μl) and Tetracycline (12 μg/μl) in a 2 ml 96 deep well culture plate. Cultures are incubated at 37° C. overnight (12-18 hrs) with shaking at 220 rpm. The next morning, the overnight culture is diluted into 800 μl of distilled water using a 96 pin replicator. 20 μl of diluted overnight culture from each well of the 96-well plate is plated onto one Nunc square plate containing LB media plus Gentamycin, Kanamycin and Tetracycline. Plates are incubated at 37° C. overnight. The next day, two Mantis 384-well output plates with 60 μl of LB plus Gentamycin (14 μg/μl) and Kanamycin (100 μg/μl) in each well are prepared, and 8 colonies from each transformation plate are picked into each well of the output plate by the Mantis colony picker. The output plates are incubated at 37° C. overnight.

Blue-White Colony QC:

Cultures in the output plate are replicated onto a LB/Bluo-Gal agar plate using a 384 pin replicator, and plates are incubated at 37° C. for at least 1 to 2 days or until the blue color developed. The blue and white colonies are analyzed using the Alpha FluorChem 8100. Wells which have nothing growing or have either light or blue colonies are failed for the next procedure. One passed colony from each clone is selected and rearrayed from the 384-well output plate into a 96-well 2 ml deep well plate containing 900 μl of 2×YT media plus Kanamycin 50 μg/μl and Gentamycin 7 μg/μl.

Bacmid Isolation:

The culture plate is grown for approximately 20-22 hours at 37° C. with shaking at 180 rpm. The next day, bacmid DNA is isolated using Perfectprep BAC 96 kit following the manufacturer's protocol (Eppendorf). 5 μl of purified bacmid DNA is analyzed on a 1% E-Gel 96 agarose gel.

Transfection and Amplification:

Insect Sf9 cells are grown in SF-900 SFM medium supplemented with 10% (v/v) Fetal Bovine Serum (FBS) and 1% (v/v) penicillin/streptomycin, and incubated in a spinner flask at 26° C. with constant stirring at 100 rpm. On the day of transfection, cells are counted and diluted to a final cell concentration of 5×105 cells/ml in Grace's insect unsupplemented medium. 100 μl of cells are aliquoted into each well of a 96-well flat bottom tissue culture plate, and attached to the surface of the plate at 26° C. for 1 hour. Meanwhile, in a new 96-well PCR plate, the DNA and cellfectin mixture is prepared as follows:

Mixture A: 3 μl of Grace's insect medium is added into each well of a

96-well PCR plate first, then 3 μl of purified bacmid DNA from the above step is added to each well of the same plate to mix with the medium.

Mixture B: For each transfection, 0.3 μl of Cellfectin is diluted into 5 μl of Grace's insect unsupplemented medium.

After adding mixture B to mixture A, the DNA:Cellfectin mixture is incubated at room temperature for 45 to 60 minutes. After 45 to 60 minutes of incubation time, for each transfection, 50 μl of Grace's insect medium is added to the mixture of A and B. Meanwhile, Sf9 cells are washed once with 100 μl of Grace's insect medium, and finally replaced with the diluted mixture A and B (about 60 μl volume). Cells are incubated in 26° C. for 5 hours. After incubation, the supernatant which contains the transfection mixtures is removed, and is replaced with 100 μl of SF-900 SFM medium containing 10% FBS and 1% (v/v) penicillin/streptomycin. Cells are incubated at 26° C. for another 72 hours. At 72 hours posttransfection, the supernatant containing the original viruses (100 μl) is harvested and transferred into a sterile round-bottom 96-well plate. The plate is sealed and stored at 4° C. in the dark. For long term storage, viruses can be stored at (−80° C.). Original viruses are amplified once to increase the virus titer. 100 μl of Sf9 cells are plated out at 1×106 cells/ml density in each well of a 96-well tissue culture plate, and allowed to attach to the surface of the plate at 26° C. for at least half an hour. 2 μl of original virus are added to the cells, and cells are incubated at 26° C. for 72 hr. At 72 h post-infection, the amplified viruses are collected into a new sterile round bottom 96-well plate, can be stored at 4° C. or −80° C., or used directly for protein expression.

Protein Expression:

Sf9 cells are counted and diluted in SF-900 II SFM medium containing 10% FBS+1% penicillin/streptomycin to a final cell density of 2×106 cells/ml. 600 μl of Sf9 cells are aliquoted into each well of a 96-deep well cell culture plate, and 6 μl of the amplified viral stock are added to the wells. The plate is sealed with a Microporous sealing film which allows compressed air to permeate during incubation, and is loaded into the Higro™ cassette. The Higro™ is run at 26° C. with shaking at 450 rpm for 72 hours.

Protein Purification:

Boxes are lysed using a Harbil paint shaker for 30 seconds in 650 μL Tris lysis buffer with protease inhibitors, incubated shaking for 15 mins then lysed again for 30 secs. Lysates are clarified by centrifugation. 38 μL of glutathione-Sepharose 4B (GE Healthcare) is added, incubated at 6° C. for 1 hr with shaking, the slurries transferred to 96 well PVDF filter plates (Whatman) then washed twice with 200 μL of HEPES wash buffer 1 and twice with 200 ul HEPES wash buffer 2. Proteins are eluted with 65 μL of Elution Buffer and consolidated into 384 well plates (Greiner, polypropylene/flat-bottom).

Western QC Sample Preparation:

At the end of expression period, 50 μl of cells from each well of the deep well culture plate are transferred into a new 96-well PCR plate. Cells are spun down, lysed in the lysis buffer and ready for further analysis as whole cell lysate. After proteins are purified, 10 μl of the purified protein is transferred into a new 96-well PCR plate. 10 μls of 2×SDS sample buffer are added to each well, and boiled in a PCR machine for 10 minutes.

SDS-PAGE:

The purchased precast gels are prerun at 150 volts for 30 minutes. Each gel has 26 lanes, therefore, 10 μls of the denatured purified proteins from two rows of the 96-well plate are loaded to the same gel using a 12-channel pipetman. On the same gel, 10 μl of the pertained protein molecular weight marker and the 10 μl of standard GST proteins (10 μg/μl) are loaded onto two separate lanes. Gels are run at a constant voltage of 150 volts for 1 hour or until the bromophenol blue marker dye is near the bottom end of the gel.

Blotting:

Each nitrocellulose membrane is labeled and soaked in the transfer buffer for a few minutes along with the Whitman 3 MM paper. The precast gel is opened, a nitrocellulose membrane is placed on top of the gel, and two Whatmann 3 MM paper are placed on each side of the gel-membrane. The gel sandwich is placed on the surface of the Semi Dry blotting apparatus with the nitrocellulose membrane on top of the gel. The electroblotting is performed at a constant current 250 mA for 20 minutes for each gel sandwich. After blotting, the apparatus is dissembled, and the membranes are probed immunochemically as described as follows:
Non-specific protein binding is blocked by incubating the membrane in blocking buffer (TBS, 0.5% Tween and 5% dried milk) for 2 hours at room temperature or overnight at 4° C.
Blocking buffer is discarded, and the membrane is incubated with primary antibody (Rabbit polyclone GST, 1:5000 dilution) in Blocking buffer for 1 to 2 hours at room temperature or overnight at 4° C.
Membrane is washed with Washing buffer for three times with 15 minutes of wash for each
Membrane is incubated with second antibody (1:5000 dilution for HRP

conjugated goat antirabbit IgG) in TBS, 0.2% BSA for 1 to 2 hours at room temperature
Membrane is washed with washing buffer again for 3 times with 15 minutes of wash for each
   Developing Membrane:

After the third wash of the membrane, it is ready for developing. Excess of washing buffer from the membrane is blocked by putting it on a paper tower for 5 seconds. A small piece of RADTape is placed on the side of prestained molecular weight marker on the membrane, the position of each band on the marker is manually marked on the tape. On a clean surface of a transparency sheet, 170 μl of solution A of the SuperSignal West Pico Maximum Signal substrate is mixed with 170 μl of solution B. The membrane is placed on top of this mixture, making sure it is covered by the solution completely. The membrane is scanned in the Alpha Innotech Fluoro Chem Apparatus, and the image is saved to a database.

Western QC Data Analysis:

The Western blot image is loaded into Western Kodak 1D 3.5 software, and analyzed by the software. Based on the size of proteins on the molecular weight marker, the size of each band for each protein on the image is calculated by the software. All the data file is saved and uploaded into ProtoMine, and proteins are passed or failed Western QC based on the following criteria:

1. If the calculated molecular weight is within the 20% range of the predicted molecular weight, it has passed.

2. If the calculated molecular weight is above the 20% range of the predicted molecular weight, it has passed.

3. If the calculated molecular weight is below a 23% range of the predicted molecular weight, it has failed.

4. If a strong protein band is observed at the expected molecular weight for the GST tag, it has failed.

5. If no protein band is observed from Western blot, it has failed.

Concentration QC:

The concentrations of human proteins are measured using microarrays. Human proteins and controls are printed on S&S FAST slides. The arrays are probed with anti-GST antibody followed by Alexa Fluor 647 antibody. The protein concentrations are derived from a GST standard gradient on the array and the spot intensities of the human proteins.

As is apparent from the protocol, the protocol comprises a number of intermediate protocols or applications. Hence the protocol, from one perspective, requires the following steps: (a) clone preparation and plasmid isolation, (b) LR reaction into vectors, (c) transformation of LR into competent cells, (d) blue-white colony selection, (e) bacmid isolation, (f) transfection and amplification, (g) protein expression, (h) protein purification, (i) Western QC sample preparation, (j) SDS-Page, (k) blotting, (l) membrane development, (m) western QC data analysis, and (n) concentration QC. From another perspective, each step of the protocol comprises a sub-protocol or application. Accordingly, the electronic commerce system may display each step individually and the related products or may provide all steps together with a single page.

Additionally, a biological research reagent vendor may provide substantially all research reagents needed to perform the protocols, applications, or workflows or alternatively may provide substantially all products to perform one step of the protocol. Thus, for example, a biological research reagent provider may display the protocol to a user who may then select the necessary products to perform at least one, some, or substantially all of the steps of the protocol.

In this example, a vendor may provide substantially all reagents necessary to execute step (n) concentration QC. Accordingly, the electronic commerce system may display links to S&S FAST slide microarrays, anti-GST antibody, and Alexa Fluor 647 antibody. Additionally, the electronic commerce system may provide a link to a “concentration QC kit” for this particular protocol that includes all of the above-recited components for performing this step of the protocol.

Alternatively, a biological research reagent provider may offer an equivalent protocol for the concentration QC step. In this example, the provider may offer an equivalent protocol or equivalent products to perform the recited step. A provider may validate the equivalent protocol or equivalent products and inform the customer that equivalent results can be obtained using the substituted protocol and products.

Similarly, a vendor may provide product for substantially all of the steps (a)-(n) of the protocol. Alternatively, each step may be associated with a separate electronic representation to allow a user to view the particular applications or protocol of each step and determine whether products are needed.

While various embodiments of the present invention have been described above, it should be understood that they have been presented by way of example only, and not limitation. For example, a variety of programming languages can be used to implement the present invention, such as well-known Java programming language, C++ programming language, C programming language, C# or any combination thereof. Thus, the breadth and scope of the present invention should not be limited by any of the above-described exemplary embodiments, but should be defined only in accordance with the following claims and their equivalents.

It should also be noted that it does not matter where the databases or other data is stored physically. Networks and Internet may connect one data object to a process just as a data bus connects physical memory or non-volatile storage to a processor. Thus, in this discussion and elsewhere, where no particular mention is made of where data is stored, it is assumed not to matter and that a person of ordinary skill could easily make a suitable decision about where to store data—on a vendor's server, on a reader, at a home network server, on a third party server, etc. Thus, profile data may “follow” a user wherever the user goes. So if a user uses an inputting device *wireless or remote peripheral device (in a public place, the user's personal profile is accessible to the processes the user employs. This assumes appropriate security devices are in place to protect the user's profile data. Also note that it has been assumed in the discussions above, in most cases, that some sort of UI, such as those built into a handled organizer with a touch screen, is associated with the inputting device discussed to allow data to be displayed and entered. The UI could be part of the device to which the inputting device is attached or with which it is associated or it could be part of the device. The details of the UI are not important, except as otherwise noted, and could be of any suitable type at the discretion of a designer.

The entirety of each patent, patent application, publication, and document referenced herein, as well as material displayed on the World Wide Web (WWW), is hereby incorporated by reference. Citation of the above patents, patent applications, publications, documents, and website materials (images and text) is not an admission that any of the foregoing is pertinent prior art, nor does it constitute any admission as to the contents or date of these publications or documents. All such publications and website materials mentioned herein are incorporated herein by reference for the purpose of describing and disclosing the processes, systems and methodologies which are reported in the publications which might be used in connection with the invention. Nothing herein is to be construed as an admission that the invention is not entitled to antedate such disclosure by virtue of prior invention.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

1-182. (canceled)

183. A method for generating revenue, comprising:
providing a protocol on an electronic commerce system, wherein the protocol is viewable by a customer who accesses the electronic commerce system without monetary consideration being provided by the customer, wherein information about at least one product is associated with the protocol on the same display, wherein the product information directly or indirectly links to a purchasing function for the product.

184. The method of claim 183, wherein the protocol is an established protocol.

185. The method of claim 184, wherein the protocol is a published and copyrighted protocol.

186. The method of claim 183, wherein the protocol is not freely available to a member of the public except through accessing the electronic commerce system.

187. The method of claim 185, wherein the copyright is owned by an entity other than the entity that provides the electronic commerce system.

188. The method of claim 185, wherein the copyright is owned by an entity other than the entity that provides the product.

189. The method of claim 187, wherein the owner of the copyrighted protocol receives consideration for purchase of a product associated with the protocol.

190. The method of claim 189, wherein the consideration is monetary consideration.

191. The method of claim 183, wherein the protocol comprises a hyperlink that provides product information.

192. The method of claim 183, wherein the protocol comprises a hyperlink that links to other protocols on the same electronic commerce system site.

193. The method of claim 183, wherein the product information is provided juxtaposed with the protocol on the same display.

194. The method of claim 183, wherein the purchasing function uses a shopping cart.

195. The method of claim 194, wherein the purchasing function uses a shared shopping cart.

196. The method of claim 183, wherein the protocol is a biology protocol.

197. A method for generating revenue, comprising:
providing a plurality of protocols on an electronic commerce system, wherein the protocols are searchable and viewable by a customer who accesses the electronic commerce system, without monetary consideration being provided by the customer, wherein information about at least one product is associated with each protocol when the protocol is viewed on a display, wherein the product information directly or indirectly links to a purchasing function for the product.

198. The method of claim 197, wherein the plurality of protocols are established protocols.

199. The method of claim 197, wherein at least two of the plurality of protocols are published in a book.

200. The method of claim 197, wherein the protocols are not freely available to a member of the public except through accessing the electronic commerce system.

201. The method of claim 197, wherein the copyright to the book is owned by an entity other than the entity that provides the electronic commerce system.

202. The method of claim 197, wherein the plurality of protocols includes protocols from two or more different technologies or methodologies selected from the group consisting of: cloning (recombinant DNA technology), transfection, cell culture, electrophoresis, mass spectrometry, nucleic acid isolation and analysis, nucleic acid amplification, protein expression and analysis, flow cytometry, immunobiology, cellular assays and analysis, gene regulation, signal transduction, cellular and developmental neuroscience, or molecular neuroscience.
